SEQUENCE LISTING (1 sequence)

SEQ ID NO: 1 - DNA, 21 bp, Homo sapiens
gtggatgaagatccaacccaa

(19) United States
(12) Patent Application Publication
(10) Pub. No.: US 2019/0008961 A1
(43) Pub. Date: Jan. 10, 2019

(54) COMPOUNDS AND METHODS FOR INCREASING TUMOR INFILTRATION BY IMMUNE CELLS

(71) Applicants: THE BROAD INSTITUTE, INC., CAMBRIDGE, MA (US); PRESIDENT AND FELLOWS OF HARVARD COLLEGE, CAMBRIDGE, MA (US); THE GENERAL HOSPITAL CORPORATION, BOSTON, MA (US)

(72) Inventors: SHRIKANTA CHATTOPADHYAY, BOSTON, MA (US); STUART L. SCHREIBER, CAMBRIDGE, MA (US)

(73) Assignees: THE BROAD INSTITUTE, INC., CAMBRIDGE, MA (US); PRESIDENT AND FELLOWS OF HARVARD COLLEGE, CAMBRIDGE, MA (US); THE GENERAL HOSPITAL CORPORATION, BOSTON, MA (US)

(21) Appl. No.: 16/068,542

(22) PCT Filed: Jan. 6, 2017
(86) PCT No.: PCT/US17/12495

§ 371 Date: Jul. 6, 2018

Related U.S. Application Data
(60) Provisional application No. 62/275,973, filed on Jan. 7, 2016.

(51) Int. Cl. A61K 39/395; A61P 35/00; A61K 31/40

(52) U.S. Cl. CPC A61K 39/39558; A61K 45/06; A61K 31/40; A61P 35/00

(57) ABSTRACT

Provided herein are compounds, compositions, and methods that increase the infiltration of tumor cell microenvironments by immune cells. Such methods are useful for enhancing a subject's immune response against the tumor.

### CROSS-REFERENCE TO RELATED APPLICATION

This application claims the benefit of the following U.S. Provisional Application No.: 62/275,973, filed Jan. 7, 2016, the entire contents of which are incorporated herein by reference.

### STATEMENT OF RIGHTS TO INVENTIONS MADE UNDER FEDERALLY SPONSORED RESEARCH

This work was supported by the following grants from the National Institutes of Health, Grant Nos: 2RO1-GM038627 and 5K08CA158149. The government has certain rights in the invention(s) disclosed herein.

### BACKGROUND

Cancer immunotherapy is a rapidly evolving field of treatment options due to the durable responses seen in some patients, even lasting years. However the benefits of immunotherapy are limited to a small number of patients, typically 15-30% of non-melanoma patients. Recent clinical correlative studies have identified that predictors of positive response include the presence of immune cells around the invasive edges of tumors, known as the tumor's microenvironment. The immune cells enhance the access of immunotherapeutic and immunogenic chemotherapeutic agents to the tumor to effect tumor cell death. Studies indicate that stromal cells around the tumor prevent immune cells from infiltrating the tumors' microenvironment. Current methods for depletion of stromal cells and recruitment of immune cells, such as genetic engineering techniques, have not met with widespread practical success in therapeutic settings. Thus, a significant need exists for reliable and effective approaches to deplete stromal cells and increase infiltration of immune cells into the tumor microenvironment, which enables anti-tumor agents to access tumor cells for their own modalities of causing tumor cell reduction.

### SUMMARY

Provided herein are compounds, compositions, and methods that increase the infiltration of tumor cell microenvironments by immune cells. Such methods are useful for enhancing a subject's immune response against a tumor.

Disclosed herein is a combination comprising a GPX4 inhibitor and an immunotherapeutic agent and/or immunogenic chemotherapeutic agent and/or lipoxygenase inhibitor. In some embodiments, the GPX4 inhibitor is selected from RSL3, ML162, buthionine sulfoximine, or an inhibitory nucleic acid molecule. In one embodiment, the GPX4 inhibitor is RSL3. In another embodiment, the GPX4 inhibitor is buthionine sulfoximine. In some embodiments, the immunotherapeutic agent is selected from a CTLA4, PDL1 or PD1 inhibitor. In certain embodiments, the CTLA4 inhibitor is ipilimumab, the PD1 inhibitor is pembrolizumab or nivolumab, and the PDL1 inhibitor is atezolizumab or durvalumab. In some embodiments, the immunogenic chemotherapeutic agent is selected from anthracycline, doxorubicin, cyclophosphamide, paclitaxel, docetaxel, cisplatin, oxaliplatin or carboplatin. In some embodiments, the lipoxygenase inhibitor is a 15-lipoxygenase inhibitor. In some embodiments, the 15-lipoxygenase inhibitor may be selected from PD146176, ML351, LOXBlock-1, LOXBlock-2, or LOXBlock-3.

In some aspects, the GPX4 inhibitor, the immunotherapeutic agent and the immunogenic chemotherapeutic agent and the lipoxygenase inhibitor are each, independently and optionally, in the form of a pharmaceutically acceptable salt. In one aspect, the GPX4 inhibitor is a pharmaceutically acceptable salt of RSL3. In another aspect, the GPX4 inhibitor is a pharmaceutically acceptable salt of buthionine sulfoximine. In some aspects, the GPX4 inhibitor is in the form of a pharmaceutical composition comprising a pharmaceutically acceptable carrier. In some aspects, the immunotherapeutic agent is in the form of a pharmaceutical composition comprising a pharmaceutically acceptable carrier. In some aspects, the immunogenic chemotherapeutic agent is in the form of a pharmaceutical composition comprising a pharmaceutically acceptable carrier.

Disclosed herein is a method of inducing one or more stromal cells' death, the method comprising contacting the stromal cells with an effective amount of a GPX4 inhibitor. In one embodiment, the stromal cells are mesenchymal cells. In one embodiment, the stromal cells are cancer-associated fibroblasts. In some embodiments, the stromal cells are derived from breast tissue, thymic tissue, bone marrow tissue, bone tissue, dermal tissue, muscle tissue, respiratory tract tissue, gastrointestinal tract tissue, genitourinary tissue, central nervous system tissue, peripheral nervous system tissue, and reproductive tract tissue. In some embodiments, the stromal cells are present in one or more tumor cells' microenvironment. In one embodiment, the tumor cells can be triple negative breast cancer (“TNBC”) cells or pancreatic cancer cells. In some embodiments, the stromal cell death is by ferroptosis.

In some aspects, the GPX4 inhibitor is selected from RSL3, ML162, buthionine sulfoximine, and an inhibitory nucleic acid molecule. In one aspect, the GPX4 inhibitor is RSL3. In another aspect, the GPX4 inhibitor is buthionine sulfoximine. In one aspect, the GPX4 inhibitor is a pharmaceutically acceptable salt of RSL3. In another aspect, the GPX4 inhibitor is a pharmaceutically acceptable salt of buthionine sulfoximine. In some aspects, the GPX4 inhibitor is in the form of a pharmaceutical composition comprising a pharmaceutically acceptable carrier.

In some embodiments, the GPX4 inhibitor increases the level of one or more arachidonic acid metabolites in the stromal cells. In some embodiments, the metabolites are selected from 5-hydroxy-eicosatrienoic acid, 5-hydroperoxy-eicosatrienoic acid, 15-hydroxy-eicosatrienoic acid, 15-hydroperoxy-eicosatrienoic acid, leukotriene LTB4, LTC4, LTD4, LTE4, prostaglandin E₂, prostaglandin G2, prostaglandin F2, 5,6-epoxy-eicosatrienoic acid, 11,12-epoxy-eicosatrienoic acid, 14,15-epoxy-eicosatrienoic acid, and 14,15-dihydroxy-eicosatrienoic acid. In one embodiment, the metabolite is leukotriene LTB4. In some embodiments, the metabolites are chemoattractants for immune cells selected from lymphocytes, phagocytes, macrophages, neutrophils, dendritic cells, mast cells, eosinophils, basophils, and natural killer cells.

Disclosed herein is a method of enhancing infiltration of immune cells into a tumor cell's microenvironment, wherein the microenvironment comprises one or more stromal cells, the method comprising contacting the one or more stromal cells with a GPX4 inhibitor. In some embodiments, the GPX4 inhibitor is selected from RSL3, ML162, buthionine sulfoximine, or an inhibitory nucleic acid molecule. In one embodiment, the GPX4 inhibitor is RSL3. In another embodiment, the GPX4 inhibitor is buthionine sulfoximine. In some embodiments, the GPX4 inhibitor is in the form of a pharmaceutically acceptable salt. In one embodiment, the GPX4 inhibitor is a pharmaceutically acceptable salt of RSL3. In another embodiment, the GPX4 inhibitor is a pharmaceutically acceptable salt of buthionine sulfoximine. In some embodiments, the GPX4 inhibitor is in a pharmaceutical composition comprising a pharmaceutically acceptable carrier. In some embodiments, the immune cells are selected from lymphocytes, phagocytes, macrophages, neutrophils, and dendritic cells, mast cells, eosinophils, basophils, and natural killer cells.

In some aspects, the method further comprises increasing the level of one or more of arachidonic acid metabolites selected from 5-hydroxy-eicosatrienoic acid, 5-hydroperoxy-eicosatrienoic acid, 15-hydroxy-eicosatrienoic acid, 15-hydroperoxy-eicosatrienoic acid, leukotriene LTB4, C4, D4, E4, prostaglandin E₂, prostaglandin G2, prostaglandin F2, 5,6-epoxy-eicosatrienoic acid, 11,12-epoxy-eicosatrienoic acid, 14,15-epoxy-eicosatrienoic acid, and 14,15-dihydroxy-eicosatrienoic acid. In some aspects, the method comprises killing one or more stromal cells in the tumor cells' microenvironment.

In some aspects, the method further comprises contacting tumor cells with an immunotherapeutic agent and/orimmunogenic chemotherapeutic agent and/or lipoxygenase inhibitor. In some aspects, the contacting with the agent results in killing one or more tumor cells. In some aspects, the immunotherapeutic agent is selected from a CTLA4, PDL1 or PD1 inhibitor. In certain aspects, the CTLA4 inhibitor is ipilimumab, the PD1 inhibitor is pembrolizumab or nivolumab, and the PDL1 inhibitor is atezolizumab or durvalumab. In one aspect, the immunotherapeutic agent is pembrolizumab. In some aspects, the immunogenic chemotherapeutic agent is a compound selected from doxorubicin, cyclophosphamide, paclitaxel, docetaxel, cisplatin, oxaliplatin or carboplatin. In some aspects, the immunotherapeutic agent is in the form of a pharmaceutical composition comprising a pharmaceutically acceptable carrier. In other aspects, the immunogenic chemotherapeutic agent is in the form of a pharmaceutical composition comprising a pharmaceutically acceptable carrier.

Disclosed herein is a method of increasing a subject's responsiveness to an immunotherapeutic or immunogenic chemotherapeutic agent, the method comprising administering to the subject in need thereof an effective amount of a GPX4 inhibitor and an effective amount of an immunotherapeutic agent and/or an immunogenic chemotherapeutic agent and/or lipoxygenase inhibitor. In some embodiments, the subject has a tumor whose cellular microenvironment is stromal cell rich. In some embodiments, the administration of the GPX inhibitor results in killing one or more stromal cells in the tumor cells' microenvironment. In some embodiments, the administration of an effective amount of an immunotherapeutic agent and/or an immunogenic chemotherapeutic agent results in killing one or more tumor cells.

In some aspects, the GPX4 inhibitor is selected from RSL3, ML162, buthionine sulfoximine and a inhibitory nucleic acid molecule. In one embodiment, the GPX4 inhibitor is RSL3. In another embodiment, the GPX4 inhibitor is buthionine sulfoximine. In some aspects, the GPX4 inhibitor is in the form of a pharmaceutically acceptable salt. In one aspect, the GPX4 inhibitor is a pharmaceutically acceptable salt of RSL3. In another aspect, the GPX4 inhibitor is a pharmaceutically acceptable salt of buthionine sulfoximine. In some aspects, the GPX4 inhibitor is in a pharmaceutical composition comprising a pharmaceutically acceptable carrier. In some aspects, the immunotherapeutic agent is selected from a CTLA4, PDL1 or PD1 inhibitor. In certain aspects, the CTLA4 inhibitor is ipilimumab, the PD1 inhibitor is pembrolizumab or nivolumab, and the PDL1 inhibitor is atezolizumab or durvalumab. In one aspect, the immunotherapeutic agent is pembrolizumab. In some aspects, the immunogenic chemotherapeutic agent is a compound selected from doxorubicin, cyclophosphamide, paclitaxel, docetaxel, cisplatin, oxaliplatin or carboplatin. In some aspects, the immunotherapeutic agent is in the form of a pharmaceutical composition comprising a pharmaceutically acceptable carrier. In other aspects, the immunogenic chemotherapeutic agent is in the form of a pharmaceutical composition comprising a pharmaceutically acceptable carrier. In some aspects, the subject has breast cancer (e.g., triple negative breast cancer, etc.) and/or pancreatic cancer.

### Definitions

Unless defined otherwise, all technical and scientific terms used herein have the meaning commonly understood by a person skilled in the art of the present disclosure. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant a substance selected from a protein, a peptide, an antibody, a nucleic acid molecule, or fragments thereof, and an organic, organometallic or inorganic compound, each of which can be present as free of other substances. An agent also includes compositions, such as formulations, complexes, composites, matrices and the like, that contain one or more of these substances. An agent can be the active compound or constituent in a therapeutic setting.

By “ameliorate” is meant decrease, suppress, attenuate, diminish, arrest, or stabilize the development or progression of a disease.

In this disclosure, “comprises,” “comprising,” “containing” and “having” and the like can have the meaning ascribed to them in U.S. Patent law and can mean “ includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

As used herein, the term “detect” refers to identifying the presence, absence or amount of the analyte to be detected. One of ordinary skill in the art readily appreciates that measurement methods inherently possess a limit(s) to its lowest and highest levels of detection. Thus, an indication of not detected as used herein is not to be construed to mean the analyte is not present at all. It is simply not present between the upper or lower limits of the detection method.

By “disease” is meant any condition or disorder that damages or interferes with the normal function of a cell, tissue, or organ. In one embodiment, the disease is cancer (e.g., breast cancer).

By “effective amount” is meant the amount of an active agent required to ameliorate the symptoms of a disease relative to an untreated subject. The effective amount of active agent(s) disclosed herein for therapeutic treatment of a disease varies depending upon a number of factors, including, but not limited to, the manner of administration, the age, body weight, and general health of the subject. The attending physician or veterinarian can decide the appropriate amount and dosage regimen.

By “enhances” is meant a positive change. In one embodiment, a GPX4 inhibitor enhances immune cell infiltration of a tumor microenvironment by at least about 10%, about 25%, about 50%, about 75%, or about 100%. By “GPX4 inhibitor” is meant any agent that inhibits the activity of the enzyme glutathione peroxidase 4 (GPX4). A GPX4 inhibitor can be either a direct or indirect inhibitor. GPX4 is a phospholipid hydroperoxidase that in catalyzing the reduction of hydrogen peroxide and organic peroxides, thereby protects cells against membrane lipid peroxidation, or oxidative stress. An indirect inhibitor blocks the formation of or depletes the concentration of glutathione. A non-limiting example is buthionine sulfoximine (BSO). A direct inhibitor of GPX4 acts to prevent binding of either or both glutathione or a lipid-hydroperoxidase in the GPX4 active site. GPX4 has a selenocysteine in the active site that is oxidized to a selenenic acid by the peroxide to afford a lipid-alcohol. The glutathione acts to reduce the selenenic acid (—SeOH) back to the selenol (—SeH). Should this catalytic cycle be disrupted, cell death occurs through an intracellular iron-mediated process known as ferroptosis. Non-limiting examples of direct GPX inhibitors include RSL3 and ML162.

By “lipoxygenase” is meant an enzyme that catalyzes the addition of oxygen to a polyunsaturated fatty acid.

By “lipoxygenase inhibitor” is meant an agent that inhibits the activity of a lipoxygenase. Exemplary lipoxygenase inhibitors include PD146176 and ML351.

By “immune infiltration” of a tumor's microenvironment is meant the presence of detectable CD3(+) or CD8(+) lymphocytes either within the tumor or adjacent to the invasive edge of the tumor using the published “Immunoscore” methodology (Galon et al., Towards the introduction of the ‘Immunoscore’ in the classification of malignant tumours. J Pathol. 2014 Jan;232(2):199-209).

By “inhibitory nucleic acid” is meant a double-stranded RNA, siRNA, shRNA, or antisense RNA, or a portion thereof, or a mimetic thereof, that when administered to a cell results in a decrease (e.g., by about 10%, about 25%, about 50%, about 75%, or even about 90-100%) in the expression of a target gene. Typically, a nucleic acid inhibitor comprises at least a portion of a target nucleic acid molecule, or an ortholog thereof, or comprises at least a portion of the complementary strand of a target nucleic acid molecule. For example, an inhibitory nucleic acid molecule comprises at least a portion of any or all of the nucleic acids delineated herein.

By “marker” is meant any protein, polynucleotide, or other analyte that increases in level or activity that is associated with a disease, disorder or treatment thereof.

As used herein, the terms “microenvironment” or “tumor microenvironment” refer to an area that is the cellular environment in which the tumor exists, including surrounding blood vessels, immune cells, fibroblasts, bone marrow-derived inflammatory cells, lymphocytes, proteins, peptides, signaling molecules and the extracellular matrix.

As used herein, the term “pharmaceutically acceptable salt” refers to those salts which are, within the scope of sound medical judgment, suitable for use in contact with the tissues of subjects without undue toxicity, irritation, allergic response and the like, and are commensurate with a reasonable benefit/risk ratio. Pharmaceutically acceptable salts are well known in the art. For example, Berge et al. describes pharmaceutically acceptable salts in detail in J. Pharmaceutical Sciences (1977) 66:1-19. Pharmaceutically acceptable salts of the compounds provided herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, organic acids from which salts can be derived include, for example, acetic acid, propionic acid, glycolic acid, pyruvic acid, oxalic acid, lactic acid, trifluoracetic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

The salts can be prepared in situ during the isolation and purification of the disclosed compounds, or separately, such as by reacting the free base or free acid of the compound with a suitable base or acid, respectively. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, iron, zinc, copper, manganese, aluminum, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. Organic bases from which salts can be derived include, for example, primary, secondary, and tertiary amines, substituted amines, including naturally occurring substituted amines, cyclic amines, basic ion exchange resins, and the like, such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

As used herein, the terms “prevent,” “preventing,” “prevention,” “prophylactic treatment” and the like refer to reducing the probability of developing a disease, disorder or condition in a subject, who does not have, but is at risk of or susceptible to developing the disease, disorder or condition.

“Radiation” or “radiation therapy” refers to one of several methods, or a combination of methods, including without limitation external-beam therapy, internal radiation therapy, implant radiation, stereotactic radiosurgery, systemic radiation therapy, radiotherapy and permanent or temporary interstitial brachytherapy. The term “brachytherapy,” as used herein, refers to radiation therapy delivered by a spatially confined radioactive material inserted into the body at or near a tumor or other proliferative tissue disease site. The term is intended without limitation to include exposure to radioactive isotopes (e.g., At-211,1-131, I-125,Y-90, Re-186, Re-188, Sm-153, Bi-212, P-32, and radioactive isotopes of Lu). Suitable radiation sources for use as a cell conditioner as provided herein include both solids and liquids. By way of non-limiting example, the radiation source can be a radionuclide, such as 1-125, 1-131, Yb 169, Ir-192 as a solid source, 1-125 as a solid source, or other radionuclides that emit photons, beta particles, gamma radiation, or other therapeutic rays. The radioactive material can also be a fluid made from any solution of radionuclide(s), e.g., a solution of I-125 or I-131, or a radioactive fluid can be produced using a slurry of a suitable fluid containing small particles of solid radionuclides, such as Au-198, Y-90. Moreover, the radionuclide(s) can be embodied in a gel or radioactive micro spheres.

By “reference” is meant a standard or control condition.

By “siRNA” is meant a double stranded RNA. In some aspects, an siRNA is about 18, about 19, about 20, about 21, about 22, about 23 or about 24 nucleotides in length and has a 2 base overhang at its 3′ end. These dsRNAs can be introduced to an individual cell or to a whole animal; for example, they may be introduced systemically via the bloodstream. Such siRNAs are used to downregulate mRNA levels or promoter activity.

As used herein, the term “stromal cell” refers to non-vascular, non-inflammatory, non-epithelial connective tissue cells of any organ that surround a tumor. Stromal cells are also known as cancer-associated fibroblasts. Stromal cells support the function of the parenchymal cells of that organ. Fibroblasts and pericytes are among the most common types of stromal cells. The term “subject,” “patient” or “individual” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1 depicts at left a central tumor cell that is surrounded by oblong stromal cells. While not being bound by any theory, inhibition of GPX4 could lead to depletion of the stromal cells and allow recruitment of immune cells, shown by circular forms, to surround the tumor cell. Treatment with an immunotherapeutic or chemotherapeutic agent such as PD1/PDL1 inhibitors or carboplatin, could then effect tumor cell death.

FIG. 2A depicts a modified wound healing assay showing the migration between GFP-labeled MDA-MC-231 (MDA) cells in the presence of mesenchymal cells (MSC) and vehicle (DMSO). FIG. 2B depicts the migration between MDA cells in the presence of MSC and RSL3. FIG. 2C depicts the lack of migration of HUVEC cells in the presence of DMSO and FIG. 2D depicts the lack of migration of HUVEC cells in the presence of RSL3.

FIG. 3 depicts the level of RSL3 induced migration for MDA cells in the presence of MSC (-●-) and for HUVEC cells (-▪-).

FIG. 4A depicts the viability of certain cell types when treated with increasing concentrations of RSL3. The plot symbols are as follows: CD34 (-●-), HUVEC (-▪-), MSC (-▴-), WI38 fibroblasts (-▾-), and MDA cells (-∇-). FIG. 4B depicts the viability of certain cell types when treated with increasing concentrations of doxorubicin. The plot symbols are as follows: CD34 (-●-), HUVEC (-▪-), MSC (-▴-), WI38 fibroblasts (-▾-), and MDA cells (-∇-)

FIG. 5A illustrates the RSL3 sensitivity of patient derived breast cancer cells and cancer associated fibroblasts. FIG. 5B shows the RSL3 sensitivity of cell-lines derived from breast cancers or stroma cells.

FIG. 6A depicts knockdown of GPX4 with shRNAs. shGPX treatment reduced GPX protein levels in both MSC and MDA cells. FIG. 6B depicts the viability of MSC-shGPX (-▪-) and MDA-shGPX (-●-) cells where MSC-shGPX cell viability was reduced relative to MSC-shLacZ controls.

FIG. 7A is a graph showing that shGPX4 knockdown increased tumor volumes in MSC, MDA, or both in a co-injection model. Similarly, FIG. 7B depicts an increase in tumor weight. (*p<0.05 compared with MDA-shLac2 and MSC-shLac2 controls) MDA-shLacZ alone is also a control in this study.

FIG. 8A depicts primary tumors from either MDA-shLacZ+MSC-shLacZ (left) or MDA-shGPX4+MSC-shGPX4 (right) conditions stained with GFP immunohistochemistry (IHC) to distinguish GFP(+) MDA cells. FIG. 8B depicts similar sections of MDA-shGPX4+MSC-shGPX4 tumors stained either with GFP IHC (left) or CD11b IHC (right) to identify myeloid cells. Arrows pointing southeast indicate tumor cells, and arrows pointing northwest indicate myeloid cells. The figures show that GPX4 knockdown tumors have markedly increased myeloid infiltration.

FIG. 9A illustrates the % live Ly6g neutrophils in immune-competent mouse models with GPX4 knockdown tumors. FIG. 9B illustrates the % live CD3+T-cells in the tumors of FIG. 9A.

FIG. 10A depicts the amount of lipoxygenase 5-HETE, an arachidonic acid metabolite, in MDA-MSC co-cultures treated with RSL3 compared to a DMSO control. FIG. 10B depicts the amount of leukotriene LTB4, an arachidonic acid metabolite, in MDA-MSC co-cultures treated with RSL3 compared to a DMSO control. FIG. 10C depicts the amount of a cyclooxygenase product PGE2, an arachidonic acid metabolite, in MDA-MSC co-cultures treated with RSL3 compared to a DMSO control. FIG. 10D depicts the amount of an epoxygenase product 14,15-EET, an arachidonic acid metabolite, in MDA-MSC co-cultures treated with RSL3 compared to a DMSO control.

FIG. 11 depicts the level of arachidonic acid metabolites present in MDA and MSC cells that are the products of Lipoxygenase in FIG. 11A, Cycloxygenase in FIG. 11B and Epoxygenase in FIG. 11C. FIGS. 11D (Lipoxygenase), 11E (Cycloxygenase), and 11F (Epoxygenase) depict the effect of RSL3 on the amounts of these respective metabolites in a co-culture of MDA-MSC cells.

FIG. 12A illustrates the results of dye-labeled T-cell chemotaxis in Boyden's chambers with the lower chamber comprising cancer and stromal cell-cultures. Results when the cancer and stromal cell cancers comprise a GPX4 inhibitor (FIG. 12B), and a combination of GPX4 inhibitor and 15-lipoxygenase inhibitor (FIG. 12C) are also illustrated. FIG. 12D compares the results of the chemotaxis measurements showing a reduction in T-cell chemotaxis for both GPX4 and 15-lipoxygenase inhibitors, but an increase when GPX4 and 15-lipoxygenase inhibitors are used together.

FIG. 13 depicts the effects of inhibitors of enzymes involved in arachidonic acid metabolism in MSC cells (-●-) treated with increasing amounts of RSL3. A control of untreated MSC cells (-▪-) is shown in each plot. In FIG. 13A, MSC cells were treated with Zileuton, a known inhibitor of 5-Lipoxygenase. In FIG. 13B, MSC cells were treated with PD146176, a known inhibitor of 15-Lipoxygenase. In FIG. 13C, MSC cells were treated with TPPU, a known inhibitor of Epoxide Hydrolase. In FIG. 13D, MSC cells were treated with Indomethacin, a known inhibitor of Cycloxygenase. In FIG. 13E, MSC cells were treated with MK886, a known inhibitor of 5-Lipoxygenase Activating Protein (FLAP).

FIG. 14 depicts the effects of inhibitors of soluble epoxide hydrolases in MSC cells treated with increasing amounts of RSL3. A control of MSC cells (-●-) treated only with RSL3 is shown in each plot. FIG. 14A shows the effect of TPPU at 20 μM (-▾-) and 80 μM (-▴-) concentration. FIG. 14B shows the effect of AUDA at 80 μM (-▴-). FIG. 14C shows the effect of butyl-AUDA at 80 μM (-▴-).

FIG. 15 depicts how viability is rescued using increasing amounts of Zileuton in MSC-shGPX cells (-●-); no effect is seen in the control MSC-shLacZ cells (-▪-).

### DETAILED DESCRIPTION

Provided herein are compounds, compositions, and methods that increase the infiltration of tumor cell microenvironments by immune cells. Such methods are useful for enhancing a subject's immune response against a tumor. It has been found that agents that inhibit GPX4 enhance immune infiltration of tumors, and could enhance subject's responsiveness to immunotherapy.

### GPX4 and Cancer Immunotherapy

Cancer immunotherapy is a promising approach to the treatment of a wide variety of cancers. However, many immunotherapeutic agents known to date have not been as successful as predicted in effecting tumor cell death. Investigation into the mechanistic pathway of these agents has identified a barrier to their access to the tumor cells. Most cancers have a fibroblast-like stromal cell rich microenvironment that encapsulates the tumor and blocks other cells or agents from reaching the tumor cells. The most common type of stromal cells are cancer-associated fibroblasts that derive from mesenchymal cells (MSCs). These microenvironments are thus sparsely populated with immune cells that would enhance the access of anti-tumor agents to tumor cells, be they biologics or small molecules. Research on inhibiting the PD1 pathway leading to immune-mediated death of cancer cells using antibodies illustrates that positive response to such inhibitors is focused on tumors that have immune cell infiltration at the tumor margin. Non-responsive tumor types lack this pre-existing immune-cell rich microenvironment (see, Gajewski, T. F., Schreiber, H. & Fu, Y. X.

Innate and adaptive immune cells in the tumor microenvironment. Nat. Immunol. 14, 1014-1022 (2013)). Previous studies have also shown that depleting stromal cells from the tumor microenvironment using genetic engineering techniques increases inflammatory infiltrates and induces responsiveness to immunotherapy drugs like CTLA4 antibodies (see, e.g., Ozdemir, B. C., et al. Depletion of carcinoma-associated fibroblasts and fibrosis induces immunosuppression and accelerates pancreas cancer with reduced survival. Cancer Cell 25, 719-734(2014)). Conventional methods of identifying anti-tumor enzymatic targets using genetic manipulation of the target to influence disease outcome can be limited by identification of targets that are not oncogenic. In vitro results often do not transfer to successful clinical outcomes. For example, methods to deplete stromal cells by inhibiting the Hedgehog pathway was not accompanied by an increase in immune cell infiltration (see, Rhim, A. D., et al. Stromal elements act to restrain, rather than support, pancreatic ductal adenocarcinoma. Cancer Cell 25, 735-747 (2014)). Such techniques have not resulted in a durable method for infiltrating the tumor microenvironment with immune cells and enabling access of immunotherapeutic drugs. Desirably, therapeutic agents for the treatment of cancer could deplete stromal cells and enhance infiltration of immune cells to the tumor's microenvironment.

Disclosed herein is a method of identifying inhibitors of GPX4 using a phenotypic high-throughput screening assay. Using a thoracic bioluminescence assay that indicated levels of lung cancer metastasis, mice co-injected with luciferase-labeled MDA-MB-231 (MDA) cells and primary human MSCs in NOD-SCID mice showed a 5-fold greater metastasis than mice injected with MDA alone. Tumors were created in immunodeficient NOD-SCID mice or immune-competent mice by subcutaneous injection of 0.5 million luciferase-labeled MDA cells either alone or co-injected in a 1:3 ratio with MSCs or WI38 fibroblasts. After 10 weeks of growth, when primary tumors reached approximately 2 cm in diameter, mice were anesthetized and injected with D-luciferin. After 20 minutes (time to signal peak), bioluminescent images were obtained using the IVIS 100 imaging system (Caliper Life Sciences, Hopkinton Mass.). ROIs were defined in the thoracic region to identify lung metastases and luminescence quantified using the instrument's software. Mice with MDA+MSC tumors had ˜5-fold greater thoracic luminescence compared to mice with MDA or MDA+WI38 tumors.

To elucidate the genetic origin of MSC-MDA interactions, gene-expression profiling of MSC-MDA co-cultures were compared to cells grown apart. Results showed that the interferon pathway was upregulated in the co-cultures, which likely indicates an inflammatory response had occurred. Transcripts present in patient stroma were prioritized and using publicly available gene-expression datasets, those transcripts that correlated with poor survival in a meta-analysis of 20 whole-tumor datasets annotated with survival outcomes were chosen. To determine if these transcripts are necessary for MSC-induced metastatic behavior, shRNA knockdown experiments were performed and levels of in vitro migration of MSC+MDA co-cultures were compared to normal endothelial cells. Knockdown of 9 genes specifically inhibited MSC+MDA migration but to a small extent. Thus, targeting individual upregulated genes is insufficient to block the cell migration phenotype.

As gene regulation experiments did not lead to a sufficient indicator of MDA-MSC interactions, small molecule libraries were screened to see if compounds could be identified as modulating mechanisms of metastasis promotion by MSCs. An in vitro phenotype was identified in which GFP-labeled MDA-MB-231 (MDA) breast cancer cells migrate 3-fold faster in the presence of MSCs compared to cells cultured alone, using a modified wound-healing assay. Compounds were identified on the basis of inhibiting MSC-promoted MDA migration. To ensure specific inhibition of MSC function, compounds were counterscreened on migration of highly motile normal human umbilical vein endothelial cells (HUVEC). Most compounds, including migration inhibitors, such as migrastatin 43, inhibited HUVEC cell migration more efficiently than MSC+MDA co-cultures. However, the compound RSL3, showed selective inhibition of MSC function by inhibiting MSC-promoted MDA migration but not that of HUVEC cells.

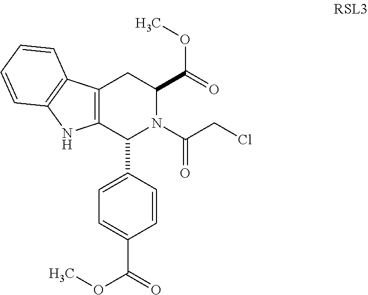 [SMILES: COC(=O)c1ccc([C@@H]2c3[nH]c4ccccc4c3C[C@@H](C(=O)OC)N2C(=O)CCl)cc1]

RSL3 is a known inhibitor of glutathione peroxidase 4 (GPX4). GPX4 is a phospholipid hydroperoxidase that in catalyzing the reduction of hydrogen peroxide and organic peroxides, thereby protecting cells against membrane lipid peroxidation, or oxidative stress. Thus, GPX4 contributes to a cell's ability to survive in oxidative environments. Inhibition of GPX4 can induce cell death by ferroptosis (see, Yang, W. S., et al. Regulation of ferroptotic cancer cell death by GPX4. Cell 156, 317-331 (2014)). RSL3 has also been demonstrated to be lethal to tumor cells in a RAS selective manner that depended upon GPX4 inhibition (see, U.S. Pat. No. 8,546,421). In knockdown studies, RSL3 selectively mediated the death of RAS-expressing cells and was identified as increasing lipid ROS accumulation. Yet, effective activity against adult stromal cells had not been demonstrated.

Pharmacokinetic studies on RSL3 in vitro showed modest stability in human plasma and liver microsomal assays and poor stability in murine counterparts. This stability is likely because of the covalent nature of RSL3's mode of action, which also explains high plasma protein binding. Given RSL's pharmacokinetic profile, intra-tumoral administration has been shown effective for RSL3 and may be a safer yet efficacious mode of administration for RSL3 (see, Yang, W. S., et al. (2014)).

Disclosed herein is a method of inducing one or more stromal cells' death by treatment with a GPX4 inhibitor. Cell death by GPX4 occurs through ferroptosis, which requires the presence of intracellular iron in a glutamate mediated cell-death process. In some embodiments, the stromal cells are MSCs. In other embodiments, the stromal cells are cancer-associated fibroblasts, other fibroblasts like WI38, breast fibroblasts (e.g., Hs578Bst) or thymic fibroblasts (e.g., Hs67). The stromal cells can be present in the tumor cells' microenvironment, and exemplary tumor cell types include triple negative breast cancer cells and pancreatic cancer cells.

The stromal cells can be derived from numerous body tissue types, including, but not limited to, breast tissue, thymic tissue, bone marrow tissue, bone tissue, dermal tissue, muscle tissue, respiratory tract tissue, gastrointestinal tract tissue, genitourinary tissue, central nervous system tissue, peripheral nervous system tissue, reproductive tract tissue.

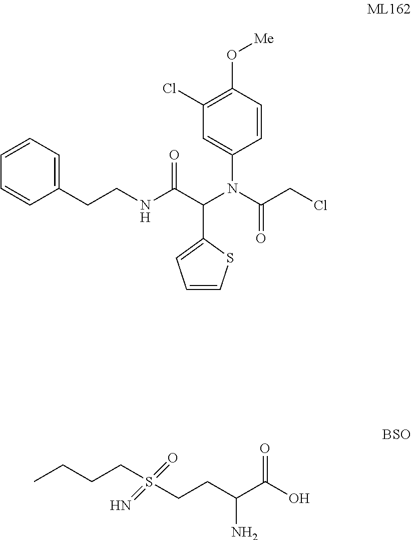 [SMILES: COc1ccc(N(C(=O)CCl)C(C(=O)NCCc2ccccc2)c2cccs2)cc1Cl ; CCCCS(=N)(=O)CCC(N)C(=O)O]

In addition to RSL3, a small molecule inhibitor ML162 has been identified as a direct inhibitor of GPX4 that induces ferroptosis (see, Dixon et al., Human Haploid Cell Genetics Reveals Roles for Lipid Metabolism. ACS Chem. Bio. 10, 1604-1609 (2015)). Indirect inhibition of GPX4 by depleting glutathione has been observed using buthionine sulfoximine (BSO) (see, Griffith, O. W. & Meister, A. Potent and specific inhibition of glutathione synthesis by buthionine sulfoximine (S-n-butyl homocysteine sulfoximine). J Blot Chem 254, 7558-7560 (1979)). BSO has human clinical safety data from previous clinical trials, which enables its use in advanced animal experimentation. Using the methods described herein, these inhibitors have also been demonstrated to effect stromal cell death but with lower potencies than that of RSL3.

In some embodiments, the methods described herein involve inhibiting the viability of stromal cells while not significantly altering the viability of tumor cells or control (non-tumor) cells. For example, the death of MSC and WI-38 fibroblasts can be induced by GPX4 inhibition while sparing MDA cancer cells and HUVEC endothelial cells. Further delineation of GPX4 inhibition resulting in selective stromal cell death included GPX4 knockdown studies using MDA and MSC cells with a short hairpin RNA (shGPX) compared to a control of MDA and MSC cells having a non-targeting hairpin (shLacZ). The knockdown resulted in lower GPX4 protein levels in both MDA and MSC cells. Yet, increased cell death was observed only in MSC cells. In addition, murine studies were performed by injecting co-cultures of MDA cells expressing shLacZ or shGPX mixed with MSCs expressing shLacZ or shGPX into the mammary fat pads of immunodeficient NOD-SCID mice, and the tumor growth was measured after 8 weeks. Greater tumor volumes were observed when GPX4 was knocked down in MSCs, in MDA cells or both. Inducing stromal cell death allowed tumor cells to grow, indicating that normal stromal cells may act to restrain tumor growth.

Microscopy of the primary tumors using stains for GFP expression to identify MDA cells indicated a difference between tumors from control conditions (MDA-shLacZ+MSC-shLacZ) versus tumors from GPX4 knockdown conditions (MDA-shGPX4+MSC-shGPX4). The increase in interstitial edema in the shGPX tumors and in GFP negative CD11b positive infiltrates (myeloid cells) demonstrated significant tumor inflammation. The inflammation was observed even where GPX4 was only knocked down in one cell-type (MDA cancer cells or MSCs). The cell death selectivity seen in these GPX4 models were then elucidated by investigation of RSL3′s mode of action within stromal cells.

Through mining gene-expression profiles of the NCI CTDD dataset, which is a dataset of 480 compounds (including RSL3) tested on ˜800 cancer cell-lines, several genes were identified to be specifically associated with RSL3 activity, but not that of other oxidative compounds. These genes were involved in arachidonic acid metabolism. Arachidonic acid is oxidatively metabolized by three groups of enzymes as shown below in Scheme 1. GPX4 inhibition results in a buildup of these metabolites, which are known to cause cell death through ferroptosis (see, Angeli et al., Inactivation of the ferroptosis regulator Gpx4 triggers acute renal failure in mice. Nat Cell Biol. 2014 Dec.; 16(12):1180-91). Confirmation of GPX4 inhibition's role in cell death has been confirmed by studies showing stromal cells can be rescued by ferrostatin (specific for preventing ferroptosis) and Zileuton (specific for the arachidonic acid lipoxygenase enzyme).

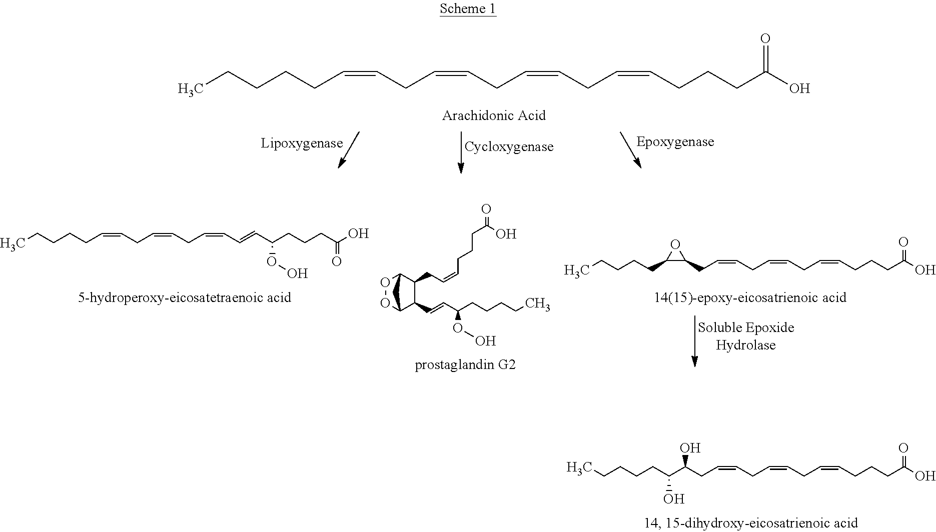 [SMILES: CCCCC/C=C\C/C=C\C/C=C\C/C=C\CCCC(=O)O ; CCCCC/C=C\C/C=C\C/C=C\C=C\[C@H](CCCC(=O)O)OO ; CCCCC[C@H](/C=C/[C@@H]1[C@H](C/C=C\CCCC(=O)O)[C@@H]2C[C@H]1OO2)OO ; CCCCC[C@H]1O[C@H]1C/C=C\C/C=C\C/C=C\CCCC(=O)O ; CCCCC[C@@H](O)[C@@H](O)C/C=C\C/C=C\C/C=C\CCCC(=O)O]

To determine whether RSL3 alters the level of arachidonic acid metabolites, unbiased lipid metabolite profiling of MDA-MSC co-cultures treated with RSL3 compared with the vehicle control DMSO was performed. In co-cultures treated with RSL3, an increase occurred in lipoxygenase products like 5-hydroxyeicosatrienoic acid and leukotrienes like leukotriene B4 (LTB4). LTB4 is known as a chemoattractant for neutrophils, a type of white blood cell, and for T cells (see Goodarzi K. et al, Leukotriene B4 and BLT1 control cytotoxic effector T cell recruitment to inflamed tissues. Nature Immunology, 2003 Oct.; 4(10):965-73.). The levels of other arachidonic acid metabolites were also increased, such as 5-hydroxy-eicosatrienoic acid, 5-hydroperoxy-eicosatrienoic acid, leukotriene LTB4, prostaglandin E₂, prostaglandin G2, 14,15-epoxy-eicosatrienoic acid, and 14,15-dihydroxy-eicosatrienoic acid (see, Scheme 1). In some embodiments, the arachadonic acid metabolites present upon treatment with RSL3 include 5-hydroxy-eicosatrienoic acid, 5-hydroperoxy-eicosatrienoic acid, 15-hydroxy-eicosatrienoic acid, 15-hydroperoxy-eicosatrienoic acid, leukotriene LTB4, LTC4, LTD4, LTE4, prostaglandin E₂, prostaglandin G2, prostaglandin F2, 5,6-epoxy-eicosatrienoic acid, 11,12-epoxy-eicosatrienoic acid, 14,15-epoxy-eicosatrienoic acid, and 14,15-dihydroxy-eicosatrienoic acid. In some embodiments, these arachidonic acid metabolites are chemoattractants for immune cells selected from lymphocytes, phagocytes, macrophages, neutrophils, dendritic cells, mast cells, eosinophils, basophils, and natural killer cells.

Studies on MSCs with known inhibitors of specific enzymes involved in the arachidonic acid metabolism pathway indicated that increasing levels of RSL3 did result in stromal cell death from 5- and 15-lipoxygenase metabolites. The Zileuton inhibitor of 5-lipoxygenase prevented RSL3-mediated cell death because the enzyme was not able to produce its normal metabolites. The RSL3 was not able to overcome the inhibitory effects of Zileuton so no buildup of metabolites was generated. Similarly, PD146176 inhibited 15-lipoxygenase from creating its metabolites such that RSL3 did not cause stromal cell death. While not wishing to be bound by any theory, these findings support the concept that RSL3 is toxic to stromal cells due to causing an increase in arachidonic acid metabolites.

Previous studies have demonstrated that current immunotherapy regimens are only effective in tumors that contain a pre-existing T-cell inflamed microenvironment. The majority of tumors lack inflammation/immune cell infiltration in their microenvironment and appear to escape immune attack through immune cell exclusion (see, Gajewski, T. F., Schreiber, H. & Fu, Y. X. Innate and adaptive microenvironment. Nat Immunol 14, 1014-1022 (2013). Preclinical studies to investigate why most tumors lack immune infiltrates have found that stromal cells in the tumor microenvironment serve as a barrier and exclude immune cells from interacting with tumor cells. Earlier studies in immunocompetent mouse models have found that depleting stromal cells from the tumor microenvironment using genetic engineering techniques increases inflammatory infiltrates and induces responsiveness to immunotherapy drugs like CTLA4 antibodies (see, Ozdemir, B. C., et al. Depletion of carcinoma-associated fibroblasts and fibrosis induces immunosuppression and accelerates pancreas cancer with reduced survival. Cancer Cell 25, 719-734(2014)). The presently described methods not only effectively kill stromal cells but allow the infiltration of immune cells, such as T-cells, with the release of chemokines like LTB4. The immune rich microenvironment thus enables access to the tumor by immunotherapeutic and immunogenic chemotherapeutic agents to cause tumor cell death (see, FIG. 1).

To this point, the GPX4 knockdown studies in mice described above indicate another role that the arachidonic acid metabolites play in the tumor microenvironment. In addition to effecting stromal cell death, the metabolites, such as leukotriene B4, recruit immune cells to the microenvironment. In some embodiments, these chemoattractants and chemokines are selected from LTB4, LTC4, LTD4, LTE4, 5,6-EET, 11,12-EET, 14,15-EET, PGE2, PGF2, PGG2, 5-HETE, 15-HETE, and 12-HETE.

Disclosed herein is a method of enhancing infiltration of immune cells into a tumor cell's microenvironment, wherein the microenvironment comprises one or more stromal cells, the method comprising contacting the one or more stromal cells with a GPX4 inhibitor, alone or in combination with a lipoxygenase inhibitor. In some embodiments, the GPX4 inhibitor is selected from RSL3, ML162, buthionine sulfoximine and a inhibitory nucleic acid molecule. In one embodiment, the GPX4 inhibitor is RSL3. In another embodiment, the GPX4 inhibitor is buthionine sulfoximine. The immune cells can be selected from lymphocytes, phagocytes, macrophages, neutrophils, and dendritic cells, mast cells, eosinophils, basophils, and natural killer cells. The method can include killing one or more stromal cells in the tumor cells' microenvironment. As described above, stromal cell death can be effected by increasing the level of one or more arachidonic acid metabolites selected from 5-hydroxy-eicosatrienoic acid, 5-hydroperoxy-eicosatrienoic acid, 15-hydroxy-eicosatrienoic acid, 15-hydroperoxy-eicosatrienoic acid, leukotriene LTB4, C4, D4, E4, prostaglandin E₂, prostaglandin G2, prostaglandin F2, 5,6-epoxy-eicosatrienoic acid, 11,12-epoxy-eicosatrienoic acid, 14,15-epoxy-eicosatrienoic acid, and 14,15-dihydroxy-eicosatrienoic acid.

Disclosed herein are further aspects of the method of enhancing infiltration of immune cells that includes contacting tumor cells with an immunotherapeutic agent or immunogenic chemotherapeutic agent. In some embodiments, the agent results in the killing of one or more tumor cells. In some embodiments, the immunotherapeutic agent is selected from a CTLA4, PDL1 or PD1 inhibitor. In some embodiments, the immunotherapeutic agent can be selected from CTLA4 inhibitor such as ipilimumab, a PD1 inhibitor such as pembrolizumab or nivolumab or a PDL1 inhibitor such as atezolizumab or durvalumab. In one embodiment, the immunotherapeutic agent is pembrolizumab. In other embodiments, the immunogenic chemotherapeutic agent is a compound selected from anthracycline, doxorubicin, cyclophosphamide, paclitaxel, docetaxel, cisplatin, oxaliplatin or carboplatin.

Disclosed herein is a method of increasing a subject's responsiveness to an immunotherapeutic or immunogenic chemotherapeutic agent, the method comprising administering to the subject in need thereof an effective amount of a GPX4 inhibitor and an effective amount of an immunotherapeutic agent and/or an immunogenic chemotherapeutic agent. In some embodiments, the method further includes administering to the subject a lipoxygenase inhibitor. In some embodiments, the subject has a tumor whose cellular microenvironment is stromal cell rich. In some embodiments, the administration of the GPX inhibitor results in killing one or more stromal cells in the tumor cells' microenvironment. In some embodiments, the administration of an effective amount of an immunotherapeutic agent and/or an immunogenic chemotherapeutic agent results in killing one or more tumor cells. In this method, the GPX inhibitor, immunotherapeutic agent, lipoxygenase inhibitor and immunotherapeutic agent are as described above and herein.

Provided herein is a combination comprising a GPX4 inhibitor and an immunotherapeutic agent, lipoxygenase inhibitor, or immunogenic chemotherapeutic agent.

In some embodiments, the GPX4 inhibitor is selected from RSL3, ML162, buthionine sulfoximine and an inhibitory nucleic acid molecule. In one embodiment, the GPX4 inhibitor is RSL3. In another embodiment, the GPX4 inhibitor is ML162. In another embodiment, the GPX4 inhibitor is buthionine sulfoximine. In another embodiment, the GPX4 inhibitor is an inhibitory nucleic acid molecule, such as, but not limited to, short hairpin RNA. In some embodiments, the immunotherapeutic agent can be selected from CTLA4 inhibitor such as ipilimumab, a PD1 inhibitor such as pembrolizumab or nivolumab or a PDL1 inhibitor such as atezolizumab or durvalumab. In other embodiments, the immunogenic chemotherapeutic agent is a compound selected from anthracycline, doxorubicin, cyclophosphamide, paclitaxel, docetaxel, cisplatin, oxaliplatin or carboplatin. In one embodiment, the GPX4 inhibitor is buthionine sulfoximine and the immunotherapeutic agent is a PDL1 antibody. In another embodiment, the GPX4 inhibitor is buthionine sulfoximine and the immunotherapeutic agent is carboplatin.

In some embodiments, a combination of the invention comprises a GPX inhibitor and a lipoxygenase inhibitor (“LOi”). In some embodiments, the lipoxygenase inhibitor may be a 15-lipoxygenase inhibitor. In some embodiments, the lipoxygenase inhibitor is selected from PD147176 and/or ML351. The 15-lipoxygenase inhibitor may be any lipoxygenase inhibitor disclosed in Sadeghian, H. et al., Expert Opinion on Therapeutic Patents, 26:1, 65-88, (2015), hereby incorporated by reference in its entirety, particularly in relation to disclosure of specific 15-lipoxygenase inhibitors.

The GPX4 inhibitors, lipoxygenase inhibitors, immunotherapeutic agents and immunogenic chemotherapeutic agents disclosed herein can be present as a single compound or biologic, or be in the form of a pharmaceutically acceptable salt and/or pharmaceutical composition that includes a pharmaceutically acceptable carrier. Pharmaceutically acceptable carriers and excipients include inert solid diluents and fillers, diluents, including sterile aqueous solution and various organic solvents, permeation enhancers, solubilizers and adjuvants. Other components of a pharmaceutical composition as described herein include, dispersion or suspension aids, surface active agents, isotonic agents, thickening or emulsifying agents, preservatives, solid binders, lubricants and the like, as suited to the particular dosage form desired. In some embodiments, a pharmaceutical composition described herein includes a second active agent such as an additional therapeutic agent, (e.g., a chemotherapeutic).

In some embodiments, the GPX4 inhibitor, the immunotherapeutic agent, the lipoxygenase inhibitor, and the immunogenic chemotherapeutic agent are each, independently and optionally, in the form of a pharmaceutically acceptable salt. Thus, in one aspect, the GPX4 inhibitor is in the form of a pharmaceutically acceptable salt and the lipoxygenase inhibitor, immunotherapeutic agent and the immunogenic chemotherapeutic agent are not in salt form. In another aspect, the immunotherapeutic agent in the form of a pharmaceutically acceptable salt, and the GPX4 inhibitor, lipoxygenase inhibitor, and the immunogenic chemotherapeutic agent are not in salt form. In another aspect, the immunogenic chemotherapeutic agent is in the form of a pharmaceutically acceptable salt and the GPX4 inhibitor and the immunotherapeutic agent are not in salt form. In another aspect, the GPX4 inhibitor and the immunotherapeutic agent are each independently in the form of a pharmaceutically acceptable salt and the immunogenic chemotherapeutic agent is not in salt form. In another aspect, the GPX4 inhibitor and the immunogenic chemotherapeutic agent are each independently in the form of a pharmaceutically acceptable salt and the immunotherapeutic agent is not in salt form. In another aspect, the GPX4 inhibitor, the lipoxygenase inhibitor, the immunotherapeutic agent and the immunogenic chemotherapeutic agent are each, independently, in the form of a pharmaceutically acceptable salt. In another aspect, none of the GPX4 inhibitor, the lipoxygenase inhibitor, the immunotherapeutic agent and the immunogenic chemotherapeutic agents are in the form of a pharmaceutically acceptable salt.

In some embodiments, the GPX4 inhibitor, the lipoxygenase inhibitor, the immunotherapeutic agent and the immunogenic chemotherapeutic agent are each, independently and optionally, in a pharmaceutical composition comprising a pharmaceutically acceptable carrier. Thus, in one aspect, the GPX4 inhibitor is in the form of a pharmaceutically composition and the lipoxygenase inhibitor, the immunotherapeutic agent and the immunogenic chemotherapeutic agent are not in composition form. In some embodiments, the immunotherapeutic agent is in the form of a pharmaceutically acceptable composition, and the GPX4 inhibitor, the lipoxygenase inhibitor, and the immunogenic chemotherapeutic agent are not in composition form. In another aspect, the immunogenic chemotherapeutic agent is in the form of a pharmaceutically acceptable composition and the GPX4 inhibitor and the immunotherapeutic agent are not in composition form. In another aspect, the GPX4 inhibitor and the immunotherapeutic agent are each independently in the form of a pharmaceutically acceptable composition and the immunogenic chemotherapeutic agent is not in composition form. In another aspect, the GPX4 inhibitor and the immunogenic chemotherapeutic agent are each independently in the form of a pharmaceutically acceptable composition and the immunotherapeutic agent is not in composition form. In another aspect, the lipoxygenase inhibitor, the GPX4 inhibitor, the immunotherapeutic agent and the immunogenic chemotherapeutic agent are each, independently, in the form of a pharmaceutically acceptable composition. In another aspect, none of the lipoxygenase inhibitor, the GPX4 inhibitor, the immunotherapeutic agent and the immunogenic chemotherapeutic agents are in the form of a pharmaceutically acceptable composition.

### Combinations and Administration Timing

GPX4 inhibitors (e.g., RSL3, ML162 and buthionine sulfoximine) may be administered alone or in combination. When administered as a combination, the therapeutic agents can be formulated as separate compositions that are administered at the same time or sequentially at different times, or the therapeutic agents can be given as a single composition. The phrases “combination” or “combination therapy”, in referring to the use of a compound as described herein (e.g., GPX4 inhibitor) together with another pharmaceutical agent (e.g., immunotherapeutic agent, immunogenic chemotherapeutic agent, the lipoxygenase inhibitor), means the coadministration of each agent in a substantially simultaneous manner as well as the administration of each agent in a sequential manner, in either case, in a regimen that will provide beneficial effects of the drug combination. Coadministration includes, inter alia, the simultaneous delivery, e.g., in a single tablet, capsule, injection or other dosage form having a fixed ratio of these active agents, as well as the simultaneous delivery in multiple, separate dosage forms for each agent respectively. That is, a compound described herein and any of the agents described herein can be formulated together in the same dosage form and administered simultaneously. Alternatively, a compound as provided herein and any of the agents herein can be simultaneously administered, wherein both the agents are present in separate formulations. In another alternative, a compound as provided herein can be administered just followed by and any of the agents described above, or vice versa. In the separate administration protocol, a compound as provided herein and any of the agents described above can be administered a few minutes apart, or a few hours apart, a few days apart, or one or more weeks apart.

The administration of disclosed compounds and combinations can be in conjunction with additional therapies known to those skilled in the art in the prevention or treatment of cancer, such as radiation therapy or cytostatic agents, cytotoxic agents, other anti-cancer agents and other drugs to ameliorate symptoms of the cancer or side effects of any of the drugs. If formulated as a fixed dose, such combination products employ the disclosed compounds within suitable dosage ranges. Compounds provided herein can also be administered sequentially with other anticancer or cytotoxic agents when a combination formulation is inappropriate. As defined herein, combination therapy is not limited in the sequence of administration; disclosed compounds can be administered prior to, simultaneously with, or after administration of the other anticancer or cytotoxic agent.

In some embodiments, treatment can be provided in combination with one or more other cancer therapies, include surgery, radiotherapy (e.g., gamma-radiation, neutron beam radiotherapy, electron beam radiotherapy, proton therapy, brachytherapy, and systemic radioactive isotopes, etc.), endocrine therapy, biologic response modifiers (e.g., interferons, interleukins, and tumor necrosis factor (TNF)), hyperthermia, cryotherapy, agents to attenuate any adverse effects (e.g., antiemetics), and other cancer chemotherapeutic drugs. The other agent(s) may be administered using a formulation, route of administration and dosing schedule the same or different from that used with the compounds and agents provided herein.

### Formulations and Administration Modes

Compositions comprising GPX4 inhibitors (e.g., RSL3, ML162 and buthionine sulfoximine), alone or in combination with other agents described herein (e.g., immunotherapeutic agent, immunogenic chemotherapeutic agent, the lipoxygenase inhibitor) can be provided by oral administration, for example, drenches (aqueous or non-aqueous solutions or suspensions), tablets (e.g., those targeted for buccal, sublingual, and systemic absorption), capsules, boluses, powders, granules, pastes for application to the tongue, and intraduodenal routes; parenteral administration, including intravenous, intraarterial, subcutaneous, intramuscular, intravascular, intraperitoneal or infusion as, for example, a sterile solution or suspension, or sustained-release formulation; topical application, for example, as a cream, ointment, or a controlled-release patch or spray applied to the skin; intravaginally or intrarectally, for example, as a pessary, cream, stent or foam; sublingually; ocularly; pulmonarily; local delivery by catheter or stent; intrathecally, or nasally.

Examples of suitable aqueous and nonaqueous carriers which can be employed in pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions can also contain adjuvants such as preservatives, wetting agents, emulsifying agents, dispersing agents, lubricants, and/or antioxidants. Prevention of the action of microorganisms upon the compounds described herein can be ensured by the inclusion of various antibacterial and antifungal agents, for example, paraben, chlorobutanol, phenol sorbic acid, and the like. It can also be desirable to include isotonic agents, such as sugars, sodium chloride, and the like into the compositions. In addition, prolonged absorption of the injectable pharmaceutical form can be brought about by the inclusion of agents which delay absorption such as aluminum monostearate and gelatin.

Methods of preparing these formulations or compositions include the step of bringing into association a compound described herein and/or the chemotherapeutic with the carrier and, optionally, one or more accessory ingredients. In general, the formulations are prepared by uniformly and intimately bringing into association a compound as disclosed herein with liquid carriers, or finely divided solid carriers, or both, and then, if necessary, shaping the product.

Preparations for such pharmaceutical compositions are well-known in the art. See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 2003; Goodman and Gilman, eds., The Pharmacological Basis of Therapeutics, Tenth Edition, McGraw Hill, 2001; Remington's Pharmaceutical Sciences, 20th Ed., Lippincott Williams & Wilkins., 2000; Martindale, The Extra Pharmacopoeia, Thirty-Second Edition (The Pharmaceutical Press, London, 1999); all of which are incorporated by reference herein in their entirety. Except insofar as any conventional excipient medium is incompatible with the compounds provided herein, such as by producing any undesirable biological effect or otherwise interacting in a deleterious manner with any other component(s) of the pharmaceutically acceptable composition, the excipient' s use is contemplated to be within the scope of this disclosure.

### Dosing

GPX4 inhibitors (e.g., RSL3, ML162, buthionine sulfoximine, etc.) described herein can be delivered in the form of pharmaceutically acceptable compositions which comprise a therapeutically effective amount of one or more compounds described herein and/or one or more additional therapeutic agents (e.g., immunotherapeutic agent, immunogenic chemotherapeutic agent, the lipoxygenase inhibitor) or such as a chemotherapeutic, formulated together with one or more pharmaceutically acceptable excipients. In some embodiments, only a compound provided herein without an additional therapeutic agent is included in the dosage form. In some instances, the compound described herein and the additional therapeutic agent are administered in separate pharmaceutical compositions and can (e.g., because of different physical and/or chemical characteristics) be administered by different routes (e.g., one therapeutic is administered orally, while the other is administered intravenously). In other instances, the compound described herein and the additional therapeutic agent can be administered separately, but via the same route (e.g., both orally or both intravenously). In still other instances, the compound described herein and the additional therapeutic agent can be administered in the same pharmaceutical composition.

The selected dosage level will depend upon a variety of factors including, for example, the activity of the particular compound employed, the severity of the condition, the route of administration, the time of administration, the rate of excretion or metabolism of the particular compound being employed, the rate and extent of absorption, the duration of the treatment, administration of other drugs, compounds and/or materials used in combination with the particular compound employed, the age, sex, weight, condition, general health and prior medical history of the patient being treated, and like factors well known in the medical arts.

The dosage level can also be informed by in vitro or in vivo assays which can optionally be employed to help identify optimal dosage ranges. A rough guide to effective doses can be extrapolated from dose-response curves derived from in vitro or animal model test systems.

Actual dosage levels of the active ingredients in the pharmaceutical compositions described herein can be varied so as to obtain an amount of the active ingredient which is effective to achieve the desired therapeutic response for a particular patient, composition, and mode of administration, without being toxic to the patient. In some instances, dosage levels below the lower limit of the aforesaid range can be more than adequate, while in other cases still larger doses can be employed without causing any harmful side effect, e.g., by dividing such larger doses into several small doses for administration throughout the day.

In some embodiments, the GPX4 inhibitor is RSL3 and is administered intra- or peritumoraly twice weekly at a dose of about 100 mg/kg, which Yang, et al. has found to be efficacious. Other administration routes include intravenous injection. In some aspects, the dose may range from about 100 mg/kg to about 400 mg/kg, such as from about 100 mg/kg to about 300 mg/kg, such as from about 100 mg/kg to about 200 mg/kg, such as from about 200 mg/kg to about 400 mg/kg, and further such as about 300 mg/kg to about 400 mg/kg. The dosing schedule can last about 1 week, about 2 weeks, about 3 weeks, or about 4 weeks.

In some embodiments, the GPX4 inhibitor is BSO and dosing follows earlier clinical work with this compound. In some embodiments, oral administration of BSO as a water solution is given twice weekly. For example, the dose for a subject may range from about 0.75 g/m2 to about 1.75 g/m2, such as about 0.75 g/m2 to about 1.5 g/m2, such as about 0.75 g/m2 to about 1.25 g/m2, and further such as about 0.75 g/m2 to about 1.0 g/m2. In other aspects, the dose may be preceded by an initial bolus of about 1 g/m2 to about 3 g/m2, such as about 1.5 g/m2. One non-limiting dosing schedule would include an about 3 g/m2 initial bolus followed by an about 0.75 g/m2 given over time, such as about 24-72 hours. Another exemplary dosing schedule would include an about 3 g/m2 initial bolus followed by an about 1.0 g/m2 given over time, such as about 24-72 hours. After this initial bolus, subjects may be administered a higher dose, such as about 1.25 g/m2, about 1.5 g/m2, or about 1.75 g/m2. In another embodiment, these dosing schedules are administered using an about 1.5 g/m2 initial bolus. The dosing schedule can last about 1 week, about 2 weeks, about 3 weeks, or about 4 weeks.

Dose information for immunotherapeutic agents like pembrolizumab, nivolumab, atezolizumab or ipilimumab follow FDA approved dosing regimens. Durvalumab is currently being considered by the FDA for marketing approval and dosing will similarly follow the FDA-approved label.

In some embodiments, the immunotherapeutic agent is nivolumab and dosing follows earlier clinical work with this agent. In some embodiments, a nivolumab dose of about 3 mg/kg is administered by intravenous infusion over about 60 min. every two weeks. In some embodiments, the immunotherapeutic agent is ipilumumab and dosing follows earlier clinical work with this agent. In some embodiments, an ipilumumab dose of about 3 mg/kg is administered intravenously over about 90 minutes every three weeks for a total of 4 doses. In some embodiments, the immunotherapeutic agent is pembrolizumab and dosing follows earlier clinical work with this agent. In some embodiments, a pembrolizumab dose of about 2 mg/kg is administered as an intravenous infusion over 30 minutes every three weeks.

Dose information for immunogenic chemotherapeutic agents like carboplatin, oxaliplatin, cisplatin, doxorubicin, cyclophosphamide, paclitaxel and docetaxel follow FDA approved dosing regimens. In some embodiments, the immunotherapeutic agent is carboplatin and dosing follows earlier clinical work with this agent. In some embodiments, a carboplatin dose of about 360 mg/m2 intravenously once daily for four weeks. In some embodiments, the immunotherapeutic agent is oxaliplatin and dosing follows earlier clinical work with this agent. In some embodiments, a oxaliplatin dose of about 85 mg/m², such as about 75 mg/m², such as about 65 mg/m², intravenously over 2 hours every 2 weeks. In some embodiments, the immunotherapeutic agent is cisplatin and dosing follows earlier clinical work with this agent. In some embodiments, a cisplatin dose of 20 mg/m² is administered intravenously daily for five days per cycle. In other embodiments, a cisplatin dose of about 50 to about 70, such as about 75 to about 100 mg/m², such as about 100 mg/m², is administered intravenously once every four weeks.

In some embodiments, the immunotherapeutic agent is doxorubicin and dosing follows earlier clinical work with this agent. In some embodiments, a doxorubicin dose of about 40 to about 60 mg/m² or about 60 to about 75 mg/m² is administered intravenously once every 21 days. In some embodiments, the immunotherapeutic agent is cyclophosphamide and dosing follows earlier clinical work with this agent. In some embodiments, a cyclophosphamide dose of about 40 to about 50 mg/kg is administered intravenously in divided doses over two to five days. In other embodiments a cyclophosphamide dose of about 10 to about 15 mg/kg is administered intravenously every seven to ten days, or about 3 to about 5 mg/kg twice weekly. In some embodiments, the immunotherapeutic agent is paclitaxel and dosing follows earlier clinical work with this agent. In some embodiments, a paclitaxel dose of about 175 mg/m² is administered intravenously over three hours, or about 135 mg/m² over 24 hours, every three weeks. Other dosing schedules include an intravenous dose of about 100 mg/m² over three hours every two weeks. Other dosing schedules also include an intravenous dose of about 80 mg/m² over one hour every week. In some embodiments, the immunotherapeutic agent is docetaxel and dosing follows earlier clinical work with this agent. In some embodiments, a docetaxel dose of about 60 to about 100 mg/m², such as about 75 mg/m², is administered intravenously over one hour every three weeks.

In some embodiments the lipoxygenase inhibitor is administered orally or intraperitoritoneally. In some embodiments, the lipoxygenase inhibitor is selected from PD146176, ML351, LOXBlock-1, LOXBlock-2, or LOXBlock-3. For example, the 15-lipoxygenase inhibitor may be administered orally at a dose of about 175 mg/kg twice daily (see Sendobry S. M. et al., British Journal of Pharmacology, 1997 April; 120(7): 1199-206). Other dosing schedules include intraperitoneal administration at a dose of about 100 mg/kg (see Hung N. D. et al., British Journal of Pharmacology, 2011 March; 162(5): 1119-35). In other embodiments, the 15-lipoxygenase inhibitor is administered intraperitoneally at a dose of about 50 mg/kg (see Rai G. et al., Journal of Medicinal Chemistry, 2014 May 22; 57(10):4035-48). In other embodiments, the 15-lipoxygenase inhibitor is administered intraperitoneally at a dose of 50 mg/kg (see Yigitkanli K. et al., Annals of Neurology, 2013 January; 73(1):129-35).

### Oncologic Indications

GPX4 inhibitors (e.g., RSL3, ML162 and buthionine sulfoximine), alone or in combination with other agents (e.g., immunotherapeutic agent, immunogenic chemotherapeutic agent, the lipoxygenase inhibitor) are useful for the treatment of tumors having a microenvironment surrounding them as described herein. Cancers having a microenvironment that is stromal cell rich with little immune cell infiltration are susceptible to the methods described herein. The cancer can be in an acute phase or chronic phase. The tumor can be in the primary phase or be metastatic. In some embodiments, the cancer can be resistant or refractory to one or more other therapies. Exemplary types of tumors as described herein include breast cancer, such as triple negative breast cancer, and pancreatic cancer.

### Immunogenic Agents

Once the stromal cells surrounding the tumor have been depleted by a GPX4 inhibitor, alone or in combination with a lipoxygenase inhibitor, immune cell infiltration allows for penetration of anti-tumor agents to the tumor cell surface. One class of such agents are immunogenic agents or therapies. Immunotherapy agents can be antibodies, such as CTLA4 antibodies (see, Ozdemir, 2014) and PDL1 antibodies (e.g., pembrolizumab). These antibodies block inhibitory checkpoints (‘brakes’) on cytotoxic T cells (effector T cells) such as CTLA4, PD-L1 and PD1. Releasing the brakes on effector T cells has resulted in unprecedented durable control of cancers lasting several years in some melanoma patients (see, Ott, P. A., Hodi, F. S. & Robert, C. CTLA-4 and PD-1/PD-L1 blockade: new immunotherapeutic modalities with durable clinical benefit in melanoma patients. Clin. Cancer Res. 19, 5300-5309 (2013)). In non-melanoma patients, PD1 and PD-L1 inhibitors have similarly shown promising durable efficacy but have low response rates ranging from 15-30% in various cancer types (see, Topalian, S. L., et al. Safety, activity, and immune correlates of anti-PD-1 antibody in cancer. N. Engl. J. Med. 366, 2443-2454 (2012)). A reliable correlate of activity with response has been expression of the inhibitory ligand PD-L1, and recent evidence suggests that PD-L1 expression occurs primarily on tumor-infiltrating immune cells. Similar analyses have found that responses to PD-1 blockade are restricted to tumors that have immune cell infiltration at the tumor margin.

Other immunotherapeutic agents that can be used in the disclosed methods include toll-like receptor agonists such as poly ICLC to enhance T cell recruitment and antigen presentation by dendritic myeloid cells. In other embodiments, CD40 agonists can be used to increase myeloid recruitment to the tumor microenvironment. Radiation therapy has also been demonstrated to be an effective immunotherapeutic agent.

### Immunogenic Chemotherapeutic Agents

Infiltration of a tumor's microenvironment by immune cells as discussed herein enables immunogenic chemotherapeutic agents to reach the tumor and affect their pathway of tumor cell death. One such class of agents are platinum-based chemotherapy drugs such as carboplatin and oxaliplatin. In one embodiment, the tumor cells are contacted with carboplatin. In another embodiment, the tumor cells are contacted with anthracycline. Additional immunogenic chemotherapeutic agents useful in the methods described herein include, but are not limited to, anthracyclines, cyclophosphamide, taxanes, and platinum agents. In one embodiment, the immunogenic chemotherapeutic agent can be selected from pembrolizumab, nivolumab, ipilimumab, atezolizumab, durvalumab, carboplatin, oxaliplatin, cisplatin, doxorubicin, cyclophosphamide, paclitaxel and docetaxel. In another embodiment, the immunogenic chemotherapeutic agent can be selected from pembrolizumab, nivolumab, ipilimumab, durvalumab and atezolizumab. In another embodiment, the immunogenic chemotherapeutic agent can be selected from carboplatin, oxaliplatin, and cisplatin. In another embodiment, the immunogenic chemotherapeutic agent can be selected from doxorubicin, cyclophosphamide, paclitaxel and docetaxel.

Radiation therapy can be administered through one of several methods, or a combination of methods, including without limitation external-beam therapy, internal radiation therapy, implant radiation, stereotactic radiosurgery, systemic radiation therapy, radiotherapy and permanent or temporary interstitial brachytherapy. The term “brachytherapy,” as used herein, refers to radiation therapy delivered by a spatially confined radioactive material inserted into the body at or near a tumor or other proliferative tissue disease site. The term is intended without limitation to include exposure to radioactive isotopes (e.g. At-211 1-131,1-125, Y-90, Re-186, Re-188, Sm 153, Bi-212, P-32, and radioactive isotopes of Lu). Suitable radiation sources for use as a cell conditioner of the present invention include both solids and liquids. By way of non-limiting example, the radiation source can be a radionuclide, such as I-125, I-131, Yb-169, Ir-192 as a solid source, I-125 as a solid source, or other radionuclides that emit photons, beta particles, gamma radiation, or other therapeutic rays. The radioactive material can also be a fluid made from any solution of radionuclide(s), e.g., a solution of I-125 or I-131, or a radioactive fluid can be produced using a slurry of a suitable fluid containing small particles of solid radionuclides, such as Au-198, Y-90. Moreover, the radionuclide(s) can be embodied in a gel or radioactive micro spheres.

The practice of the presently disclosed compounds, compositions and methods employs, unless otherwise indicated, conventional techniques of molecular biology (including recombinant techniques), microbiology, cell biology, biochemistry, immunology and chemistry, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides provided herein, and, as such, may be considered in making and practicing the disclosed embodiments. The following examples are put forth so as to provide those of ordinary skill in the art with a description of how to make and use the disclosed assay, screening, and therapeutic methods, and are not intended to limit the scope of the recited claims.

### EXAMPLES

From the foregoing description, it will be apparent that variations and modifications may be made to the procedures described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

### Example 1

### Phenotypic Approach to Discovering Drug Candidates

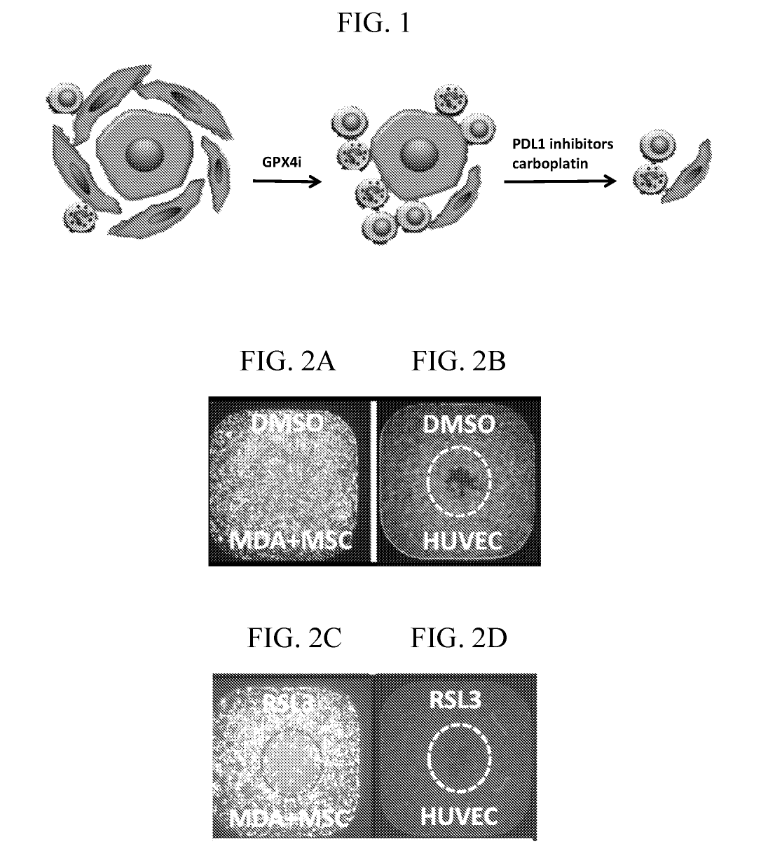

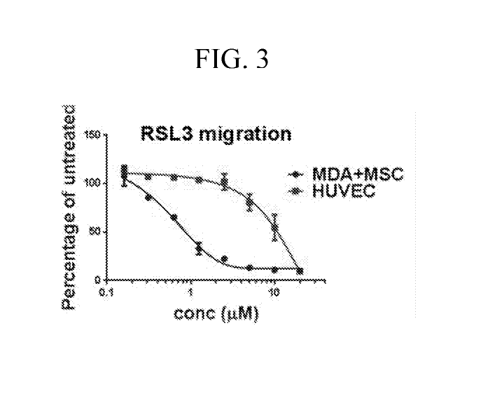

Bone marrow mesenchymal stem cells (MSCs), which home into the sites of breast cancer and are precursors of cancer-associated fibroblasts, promote breast cancer metastasis. An in vitro phenotype was developed in which GFP-labeled MDA-MB-231 (MDA-GFP) breast cancer cells migrate 3-fold faster in the presence of MSCs compared to cells cultured alone, using a modified wound-healing assay. This assay involved using bone-marrow derived mesenchymal stem cells from healthy donors (Lonza, Walkersville MDA catalog # PT-2501) and co-culturing them with MDA-GFP cells in a 3:1 ratio in commercially available 384-well cell-migration plates (Platypus Technologies, Madison WI catalog # PRO384CMACC5). When cells were seeded into these migration plates, they formed a monolayer around a circular gap (“wound”) because of the presence of a circular biodegradable gel. Once the gel dissolved, cells were allowed to migrate into the gap and the number of GFP(+) that migrated into the gap were quantified by high-throughput microscopy (IXMicro, Molecular Devices Sunnyvale Calif.) and automated image analysis (MetaXpress software, Molecular Devices). A small-molecule screen using this assay identified compounds that inhibit MSC-promoted MDA migration. To ensure specific inhibition of MSC function, compounds were counter-screened on migration of highly motile normal human umbilical vein endothelial cells (HUVEC). Most compounds, including migration inhibitors such as migrastatin 43, inhibited HUVEC cell migration more efficiently than MSC+MDA co-cultures (see FIGS. 2A, 2B, and 2D). RSL3 showed selective inhibition of MSC function by inhibiting MSC-promoted MDA migration but not that of HUVEC cells (see FIGS. 2C and 3).

### Example 2

### GPX4 as a Target for Stromal Depletion

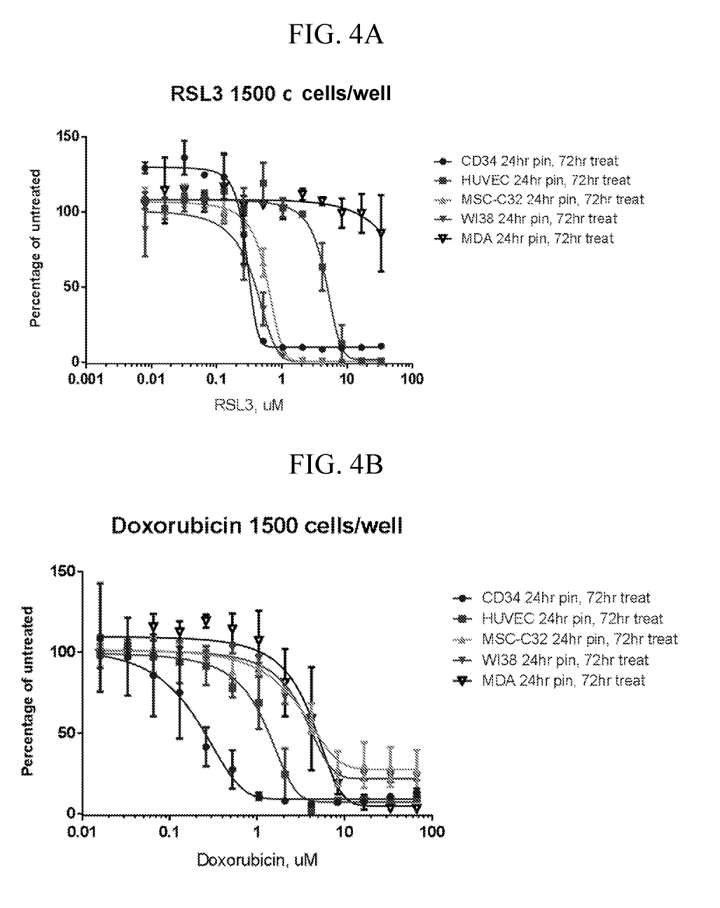

Viability measurements were made to determine whether RSL3 is toxic to MDA cells, MSCs or both. Viability was assessed by seeding cells into 384-well opaque-bottom plates (Corning, Corning N.Y. catalog #3570) then treating them 24 hours later with varying concentrations of RSL3 (added using the pin-transfer instrument CyBi-Well Vario, Cybio, Woburn Mass.) for an additional period of 72 hours. Viable cells remaining after the treatment were quantified using the Cell Titer Glo reagent (Promega, Madison Wis. catalog #G7570) that measures cellular ATP content. RSL3 was >10-fold more potent in inhibiting viability of stromal cells like MSCs and WI-38 fibroblasts compared with MDA cancer cells or HUVEC endothelial cells (see FIG. 4A). MSCs and fibroblasts are generally not sensitive to anti-neoplastic drugs and did not show enhanced sensitivity to drugs such as doxorubicin (see

FIG. 4B). Fibroblasts derived from breast specimens (Hs578Bst), thymic fibroblasts (Hs67), and CD34⁺ hematopoietic cells also exhibit loss of viability with RSL3 concentrations similar to those for MSCs and WI38 fibroblasts.

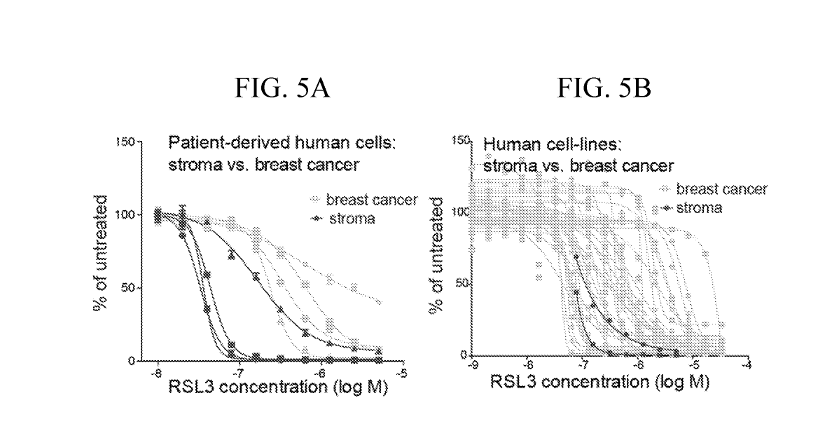

Viability measurements were also performed using primary cells derived from anonymous samples similar to Crystal, A. S., et al., Science, 346 1480-1486 (2014), hereby incorporated by reference in its entirety, particularly in relation to disclosure of cell derivation. RSL3 was shown to selective inhibit the viability of cancer-associated fibroblasts over breast cancer cells (see FIG. 5A). Similarly, stromal cell-lines were more sensitive to RSL3 than most breast cancer cell-lines (see FIG. 5B). Other known inhibitors of GPX4 include ML162 and buthionine sulfoximine (BSO).

### Example 3

### RSL3 Toxicity to MSCs Occurs via Inhibition of GPX4

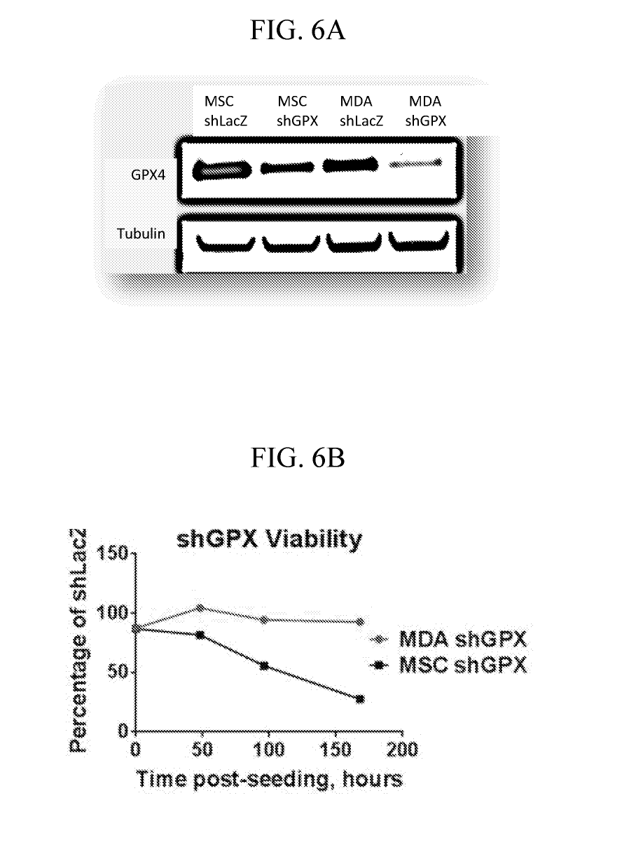

To determine whether knockdown of GPX4 would be toxic to MSCs, MSCs and MDA, cancer cells were generated to stably express short RNA hairpins targeting GPX4 (shGPX) or expressing untargeted short hairpin RNA (shLacZ). Briefly, pLKO.5 lentiviral vectors encoding short hairpin RNAs targeting either a specific sequence (GTGGATGAAGATCCAACCCAA) on the messenger RNA of GPX4 or the LacZ gene (control) were transduced into MSCs or MDA cells. After 2 days of puromycin selection to eliminate non-transduced cells, Western blot assays were performed for protein expression of GPX4 (Abcam, Cambridge Mass. antibody catalog #ab125066) and cells were seeded in 384-well plates to serially assess viability using the Cell Titer Glo reagent. In the Western blot analysis with tubulin as a control, shGPX resulted in lower GPX4 protein levels in both MDA cancer cells and MSCs relative to non-targeting hairpins (shLacZ), but lowered viability only in MSCs (see FIGS. 6A and 6B). Altogether, these studies indicate that reduction of MSC viability by RSL3 is mediated by GPX4 inhibition.

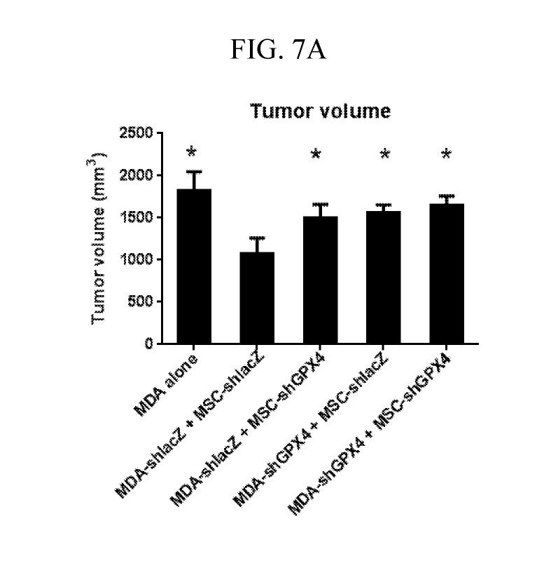

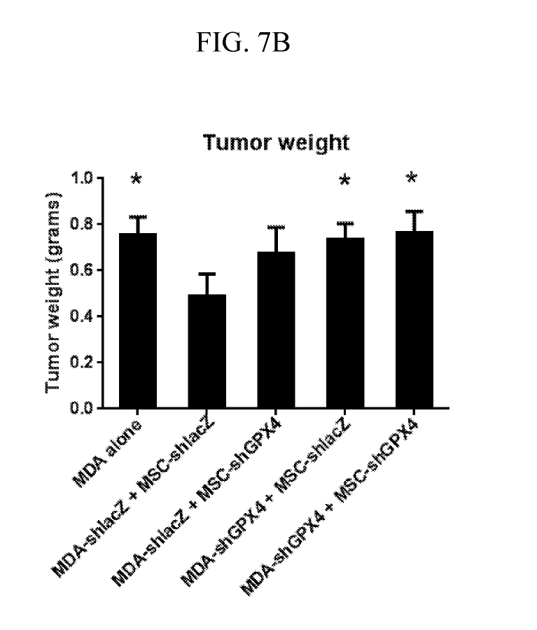

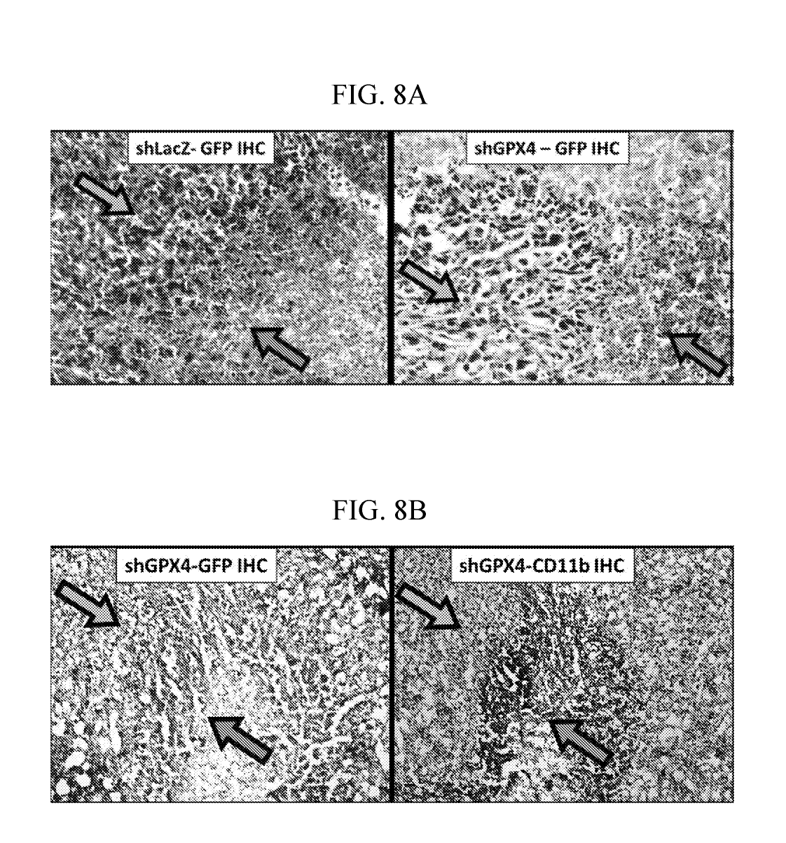

Then, 0.8 million MDA-GFP cells expressing shLacZ or shGPX mixed in a 1:3 ratio with 2.4 million MSCs expressing shLacZ or shGPX were co-injected into the mammary fat pads of immunodeficient NOD-SCID mice and tumor growth was measured after 8 weeks. Tumor volumes and weight increased when GPX4 was knocked down in MSCs, in MDA cells or both (see FIGS. 7A and 7B). Tumors were stained for GFP expression (Cell Signaling Technologies, Danvers Mass. antibody catalog #2555S) using standard immunohistochemistry to identify MDA cancer cells and differences between tumors from control conditions (MDA-shLacZ+MSC-shLacZ) and tumors from knockdown conditions (MDA-shGPX4+MSC-shGPX4) were observed. The GFP(+) cancer cells were more tightly packed in control tumors and loosely packed in shGPX tumors suggestive of interstitial edema in the latter group (see FIG. 8A). Increased GFP negative CD11b positive (Biolegend San Diego Calif., antibody catalog #101201) infiltrates around GPX4 knockdown tumors were observed indicating myeloid infiltration (see FIG. 8B). These results are consistent with GPX4 knockdown leading to increases in eicosanoid chemoattractants that require co-operative transcellular biosynthesis between MDA cells and MSCs.

### Example 4

### GPX4 Inhibition in Tumors in Immune-Competent Mice Cells Modulates Inflammatory T-cell Infiltration

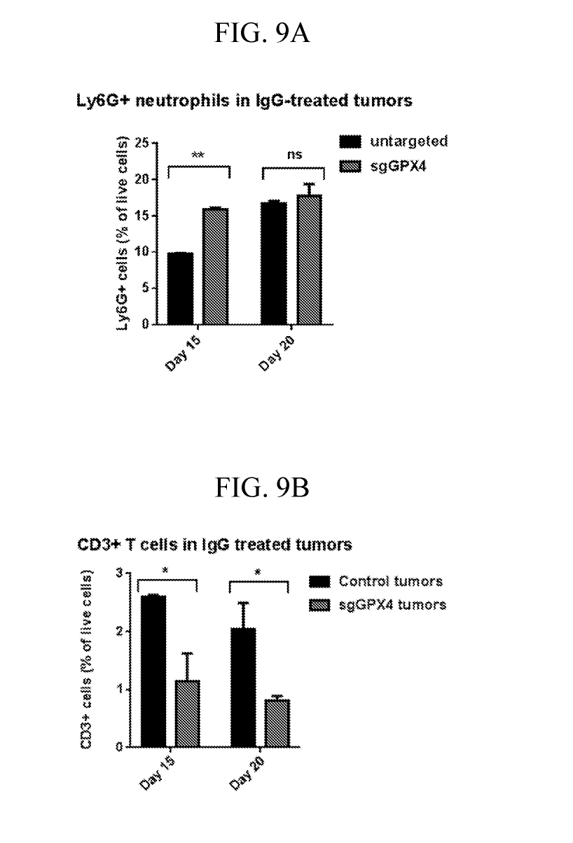

Xenografts of murine 4T1 triple negative breast cells were co-injected with stromal cells into mammary fat pads of balb/c mice. Targeting of clustered regularly interspaced short palindromic repeats of GPX4 in these tumors (“CRISPR”) was performed prior to injection and tumors were harvested 15 days and 20 days following injection. CRISPR mediated targeting of GPX4 (“sgGPX4”) in these tumor cells showed a significant increase in neutrophil recruitment measured by fluorescence-activated cell sorting (“FACS”) as compared to an untargeted control. However, T-cell infiltration was unexpectedly suppressed in sgGPX4 tumors (see FIG. 9A). Moreover, in tumors treated with PDL1 checkpoint immunotherapy, intra-tumoral T-cell infilitration showed an even greater decrease in sgGPX4 tumors (see FIG. 9B). Moreover, this T-cell suppression was not related to increased neutrophilic infilitration of the tumors because treating sgGPX4 tumors with a Ly6G antibodies that targets neutrophils did not result in increased T-cell infiltration. Therefore, the knockdown of GPX4 appears to increase non-chemokine chemo-attraction of neutrophils, but chemo-repulsion of T-cell infiltration.

### Example 5

### GPX4 Inhibition in MSCs Results in Increased Levels of Arachidonic Acid Metabolites

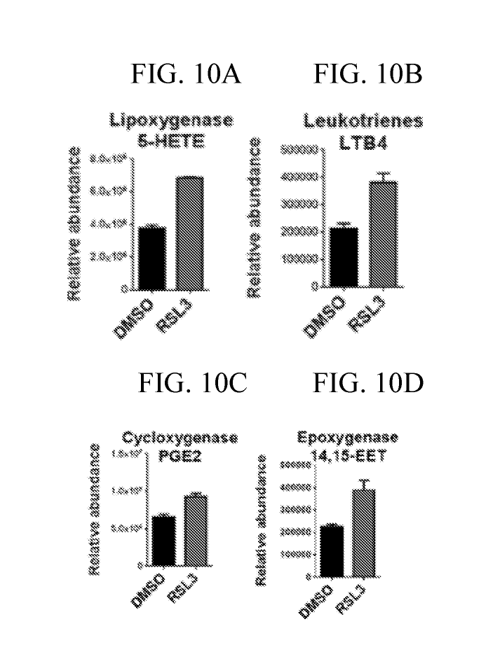

Using RSL3 as a probe, the role of GPX4 inhibition on arachidonic acid metabolism was studied. Unbiased lipid metabolite profiling of MDA-MSC co-cultures treated with RSL3 were compared with the vehicle control DMSO. Briefly, MSCs and MDA cells were either seeded alone or co-cultured in 15 cm tissue dishes. Co-cultures were either treated with 10 μM RSL3 or DMSO (vehicle control) for 6 hours. Thereafter, media was aspirated and lipids were extracted from cells using 15 mL ice-cold mass-spectrometry grade methanol and concentrated by evaporating methanol under a stream of nitrogen until a final volume of 150 μL. The endogenous charged lipid metabolites were then identified and quantified using liquid chromatography tandem mass spectrometry (LC-MS). Liquid chromatography, through the application of a number of distinct stationary phase chemistries, afforded reproducible separation of metabolites in complex mixtures on the basis of their physical properties. Mass spectrometry enabled further resolution of metabolites on the basis of mass-to-charge ratio (m/z) and quantitation over a wide linear dynamic range. Metabolites were identified by the parent ion mass (MS) and dominant product ion mass (MS/MS) on a sensitive mass spectrometer in combination with the retention time on an appropriate chromatography column. While none of these three parameters is individually sufficient to uniquely identify a metabolite in a biological sample, the three in combination provide a “tag” that marks the metabolite to permit identification and quantitation. Differences in individual metabolites were identified using the software MultiQuant (v 2.1, AB SCIEX) and Progenesis CoMet software (v 2.0, Nonlinear Dynamics). MultiQuant (v 2.1, AB SCIEX) rapidly integrates large numbers of peaks in the samples and visualize the results for inspection of quality. Targeted data were processed using MultiQuant software and compound identities were confirmed using reference standards and reference samples. Non-targeted data were processed using Progenesis CoMet software (v 2.0, Nonlinear Dynamics) to detect peaks, perform chromatographic retention time alignment, and integrate peak areas. Non-targeted metabolite LC-MS peaks were initially identified by matching measured retention times and masses to a database of >500 characterized compounds, and secondarily by matching exact masses only to a database of >40000 metabolites (Human Metabolome Database v3). Bioinformatics analyses were conducted using MetaboAnalyst 3.0. The profiling identified lipoxygenase products like leukotriene B4 (LTB4) to be significantly increased with RSL3 treatment (see FIG. 10B). Increases in lipoxygenase metabolite 5-hydroxyeicosatrienoic acid (5-HETE), cyclooxygenase metabolite prostaglandin E₂ (PGE2) and epoxygenase metabolite 14,15-epoxyeicosatrienoic acid (14,15-EET) were also observed with increasing levels of RSL3 (see FIGS. 10A, 10C, and 10D). Epoxygenase products were increased to a lesser extent and cyclooxygenase products were slightly increased.

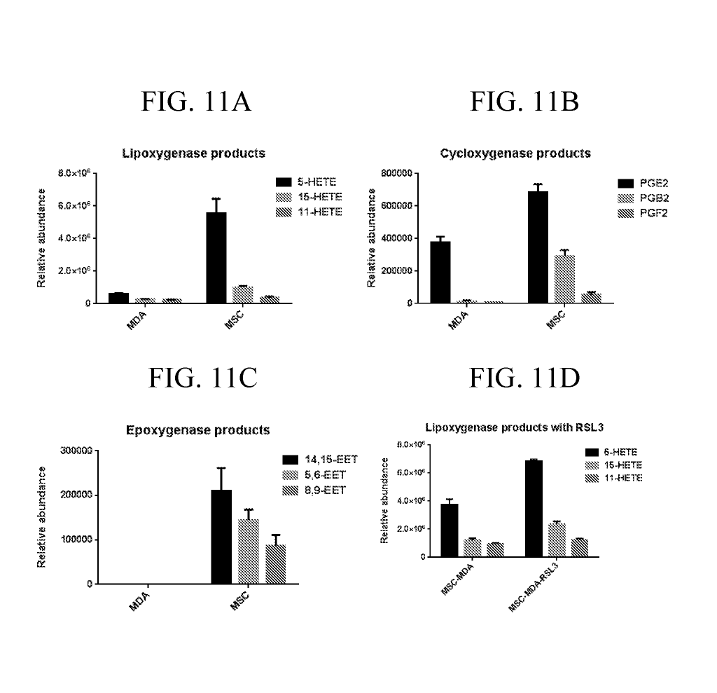

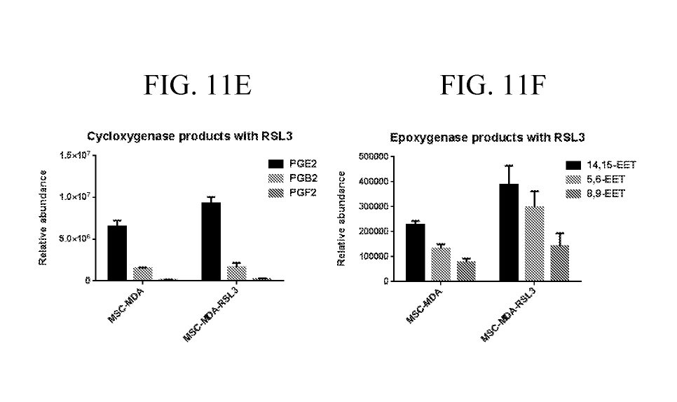

The abundance of lipoxygenase metabolites 5-HETE, 11-HETE and 15-HETE in MSCs is greater than in MDA cells (see FIG. 11A). Similarly, cyclooxygenase metabolites PGE2, PGB2 and PGF2 (see FIG. 11B) and epoxygenase metabolites 14,15-EET, 5,6-EET, and 8,9-EET are also increased in MSC cells. The levels of all these metabolites increases in both MDA and MSC cells when treated with RSL3 (see FIGS. 11D, 11E, and 11F).

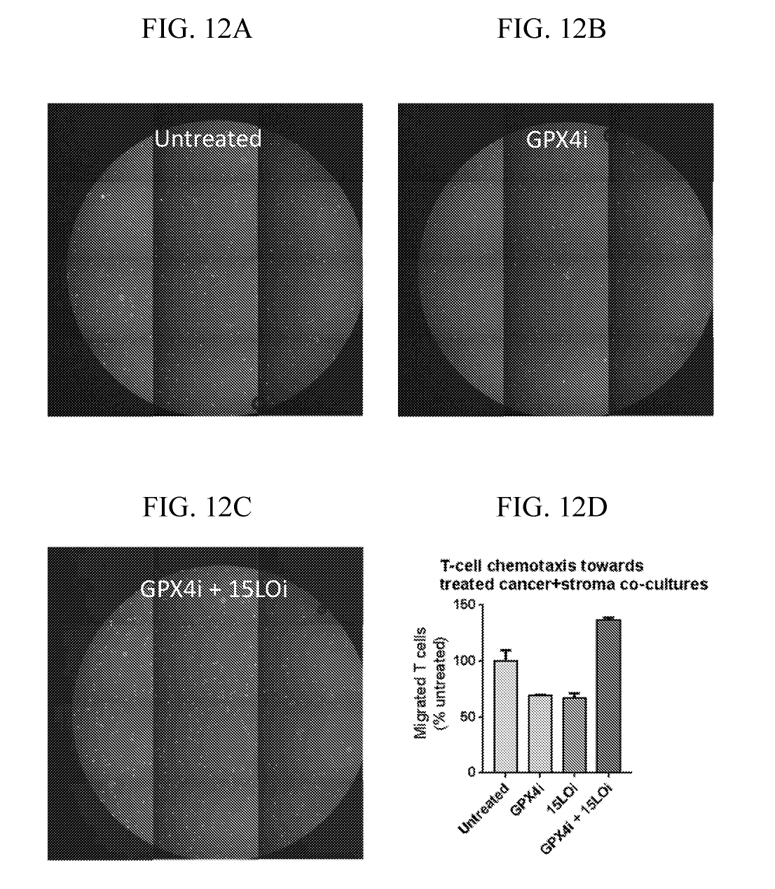

Determining whether 15-lipoxygenase products (“LOi”) contribute to T-cell chemo-repulsion occurred in a high-throughput T-cell chemotaxis assay in 96-well Boyden's chambers. Initially, the T-cells were located in the upper chamber and the lower chamber comprised cancer and stromal cell co-cultures. The chemotaxis of dye-labeled T-cells from the upper chamber to the lower chamber showed that modulation of T-cell chemotaxis was dependent on GPX4 inhibition. As shown in FIGS. 12A, 12B, 12C, and 12D, as compared with untreated co-cultures, co-cultures treated with either GPX4 inhibitors (“GPX4i”) or lipoxygenase inhibitors (“LOi”) showed reduced T-cell chemotaxis (see FIG. 12D for direct comparison) in the cell. Surprisingly, the combined treatment of co-cultures using both GPX4 and lipoxygenase inhibitors significantly increased T-cell chemotaxis although each product alone decreased chemotaxis. While not wishing to be bound by any theory, the combined inhibition of GPX4 and 15-lipoxygenase may enable stromal cells to selectively increase 5-lipoxygenase products (e.g., LTB4) which are potent chemo-attractants for T cells.

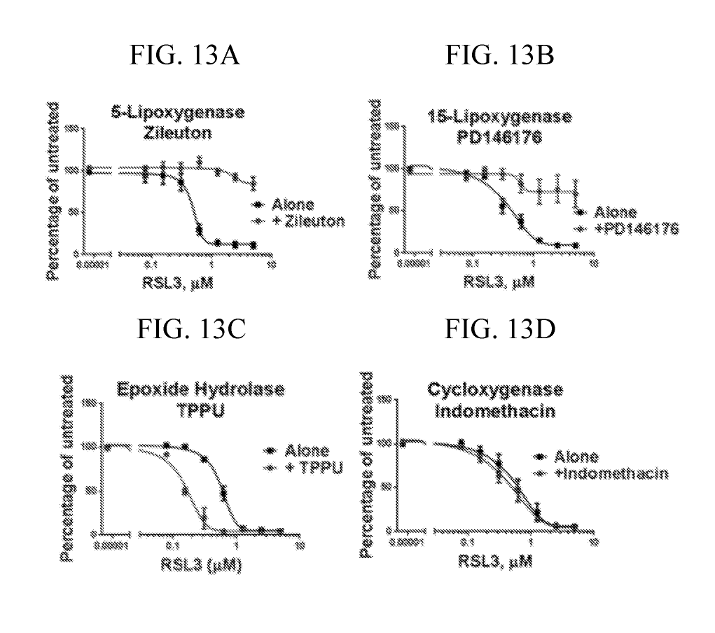

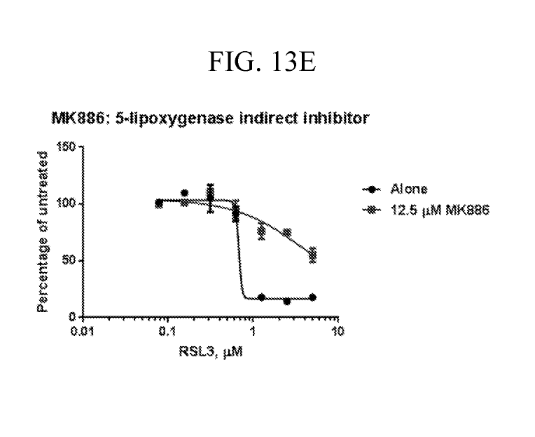

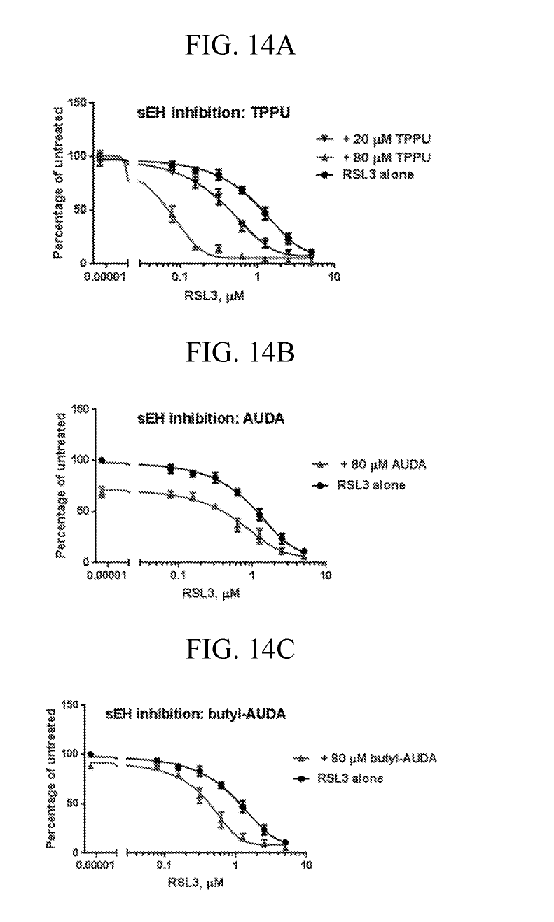

To investigate the role of RSL3's effects on MSC toxicity, MDA-MSC co-cultures were contacted by compounds that were known inhibitors of arachidonic acid metabolic enzymes. Treating the MDA-MSC cells with Zileuton, a 5-lipoxygenase inhibitor, and PD146176, a 15-lipoxygenase inhibitor, with increasing amounts of RSL3 showed that these compounds lessened the toxicity of RSL3 (see FIGS. 13A and 13B). MK886, a 5-lipoxygenase indirect inhibitor, also lessened the toxicity of RSL3 (see FIG. 13E). Cyclooxygenase inhibition with Indomethacin had little impact while an epoxide hydrolase inhibitor TPPU, that increases epoxygenase products, enhanced RSL3 effects (see FIGS. 13C and 13D). Further experiments with soluble epoxidase inhibitors TPPU, AUDA, and butyl-AUDA confirmed the increase in RSL3 mediated cell death in MSCs (see FIGS. 14A, 14B, and 14C).

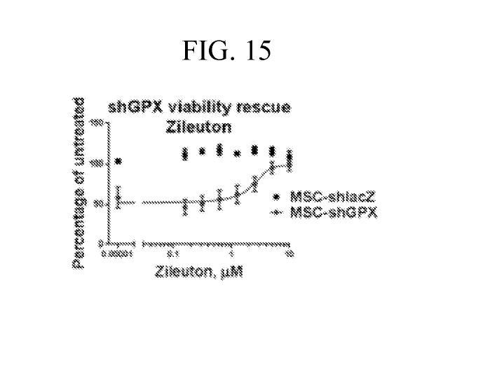

In a knockdown GPX4 experiment in MSCs, increasing amounts of Zileuton were added to MSC-shLacZ cells as a control and to MSC-shGPX cells growing in 384-well plates and their viability was measured after 5 days of growth (see FIG. 15). Despite GPX4 inhibition, the amount of MSC-shGPX cells that survived increased with higher amounts of Zileuton, illustrating that inhibition of 5-lipoxygenase resulted in a lower amount of its metabolite 5-HETE. While not wishing to be bound by any theory, these results suggest that increasing levels of arachidonic acid metabolites in MSC cells is effected with GPX4 inhibition and that this increase results in cell death of the MSC cells.

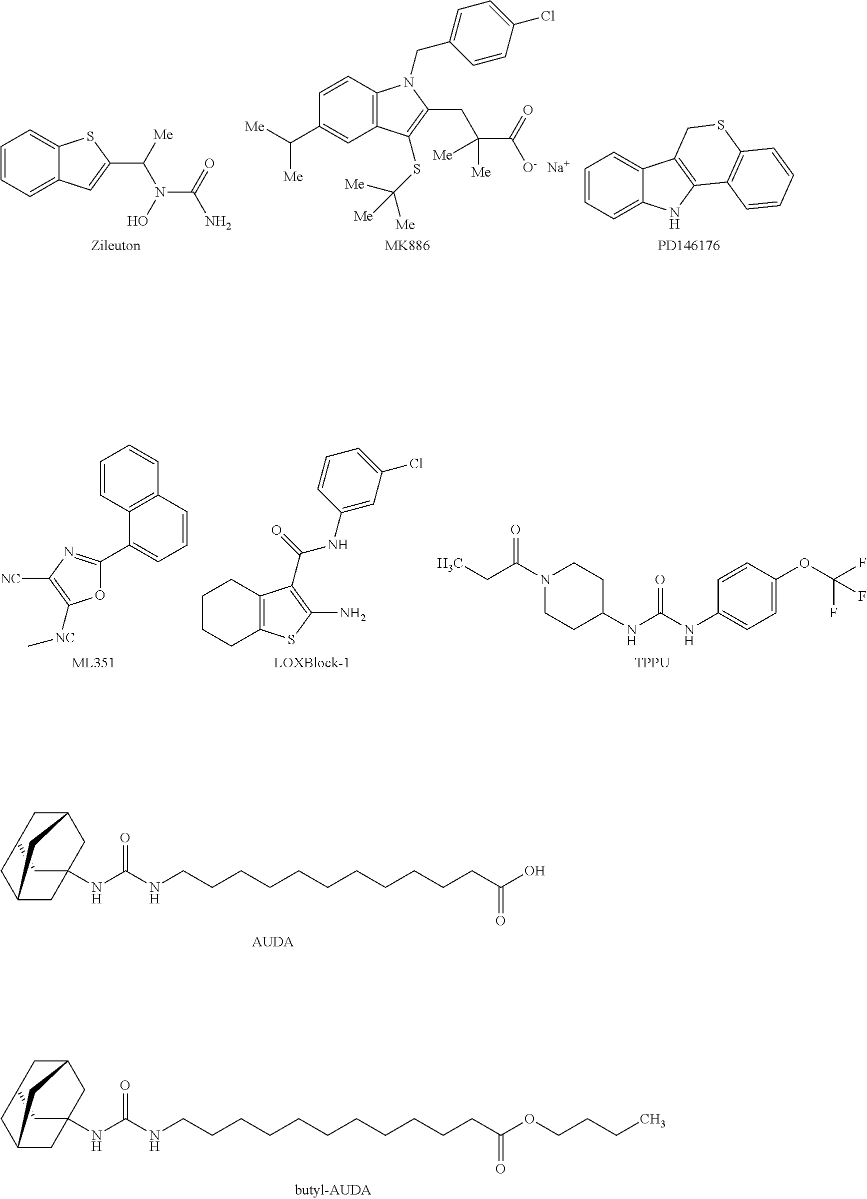

### Example 6

### Pharmacokinetic Studies of RSL3

The RSL3 in vitro plasma stability at 5 hours, plasma protein binding at 5 hours and liver microsome stability at 1 hour were determined as shown in Table 1. The following methods were used:

Plasma Protein Binding. Plasma protein binding was determined by equilibrium dialysis using the Rapid Equilibrium Dialysis (RED) device (Pierce Biotechnology, Rockford, Ill.) for both human and mouse plasma. Each compound was prepared in duplicate at 5μM in plasma (0.95% acetonitrile, 0.05% DMSO) and added to one side of the membrane (200 μL) with PBS pH 7.4 added to the other side (350 μL). Compounds were incubated at 37° C. for 5 hours with a 350-rpm orbital shake. After incubation, samples were analyzed by UPLC-MS (Waters, Milford, Mass.) with compounds detected by SIR detection on a single quadrupole mass spectrometer.

Plasma Stability. Plasma stability was determined at 37° C. at 5 hours in both human and mouse plasma. Each compound was prepared in duplicate at 5 μM in plasma diluted 50/50 (v/v) with PBS pH 7.4 (0.95% acetonitrile, 0.05% DMSO). Compounds were incubated at 37° C. for 5 hours with a 350-rpm orbital shake with time points taken at 0 hours and 5 hours. Samples were analyzed by UPLC-MS (Waters, Milford, Mass.) with compounds detected by SIR detection on a single quadrupole mass spectrometer.

Microsomal Stability. Microsomal stability was determined at 37° C. at 60 minutes in both human and mouse microsomes. Each compound was prepared in duplicate at 1 μM with 0.3 mg/mL microsomes in PBS pH 7.4 (1% DMSO). Compounds were incubated at 37° C. for 60 minutes with a 350-rpm orbital shake with time points taken at 0 minutes and 60 minutes. Samples were analyzed by UPLC-MS (Waters, Milford, Mass.) with compounds detected by SIR detection on a single quadrupole mass spectrometer.

RSL3 had modest stability in human plasma and liver microsomal assays and little stability in murine counterparts.

TABLE 1

| In Vitro Measurement | Human | Mouse |
|---|---|---|
| Plasma Stability (5 h) | 47% | <1% |
| Plasma Binding Protein (5 h) | 98.2% | N/A |
| Liver Microsome Stability (1 h) | 32.7% | <1% |

The known covalent mode of action of RSL3 in GPX4 inhibition may contribute to the high human plasma binding protein level in humans which, while not wishing to be bound by any theory, could pose a challenge to systemic delivery. However, intra-tumoral administration of RSL3 has been shown to be effective in other studies (see, Yang, W. S., et al. (2014)).

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A combination comprising a GPX4 inhibitor and an immunotherapeutic agent, a lipoxygenase inhibitor, an immunogenic chemotherapeutic agent, or combinations thereof.

2. The combination of claim 1, comprising a GPX4 inhibitor and a lipoxygenase inhibitor.

3. The combination of claim 2, wherein the GPX4 inhibitor is selected from RSL3, ML162, buthionine sulfoximine, or an inhibitory nucleic acid molecule; the immunotherapeutic agent is selected from a CTLA4, PDL1 or PD1 inhibitor; the lipoxygenase inhibitor is a 15 lipoxygenase inhibitor.

4-6. (canceled)

7. The combination of claim 6, wherein the CTLA4 inhibitor is ipilimumab, the PD1 inhibitor is pembrolizumab or nivolumab, and the PDL1 inhibitor is atezolizumab or durvalumab.

8-16. (canceled)

17. A method of inducing one or more stromal cells' death, the method comprising contacting the stromal cells with an effective amount of a GPX4 inhibitor.

18. The method of claim 17, wherein the stromal cells are mesenchymal cells or cancer-associated fibroblasts.

19. (canceled)

20. The method of claim 18, wherein the stromal cells are derived from breast tissue, thymic tissue, bone marrow tissue, bone tissue, dermal tissue, muscle tissue, respiratory tract tissue, gastrointestinal tract tissue, genitourinary tissue, central nervous system tissue, peripheral nervous system tissue, and reproductive tract tissue.

21. (canceled)

22. The method of claim 17, wherein the tumor cells are triple negative breast cancer cells or pancreatic cancer cells.

23-33. (canceled)

34. The method of claim 17, wherein the GPX4 inhibitor increases the level of one or more arachidonic acid metabolites in the stromal cells.

35-37. (canceled)

38. A method of enhancing infiltration of immune cells into a tumor cell's microenvironment, wherein the microenvironment comprises one or more stromal cells, the method comprising contacting the one or more stromal cells with a GPX4 inhibitor. the method further comprising contacting the one or more stromal cells with a GPX4 inhibitor and a lipoxygenase inhibitor.

40. A method of treating cancer in a subject, the method comprising administering to the subject with a GPX4 inhibitor and a lipoxygenase inhibitor.

41. The method of claim 40, wherein the subject has breast cancer or pancreatic cancer.

42. The method of claim 41, wherein the subject has triple negative breast cancer.

43-64. (canceled)

65. A method of increasing a subject's responsiveness to an immunotherapeutic or immunogenic chemotherapeutic agent, the method comprising administering to the subject in need thereof an effective amount of a GPX4 inhibitor and an effective amount of an immunotherapeutic agent and/or an immunogenic chemotherapeutic agent and/or one or more lipoxygenase inhibitors.

66. The method of claim 65, wherein the subject has a tumor whose cellular microenvironment is stromal cell rich.

67. The method of claim 65, wherein the administration of the GPX inhibitor results in killing one or more stromal cells in the tumor cells' microenvironment.

68. The method of claim 65, wherein the administration of an effective amount of an immunotherapeutic agent and/or an immunogenic chemotherapeutic agent results in killing one or more tumor cells.

69. The method according to claim 65, wherein said lipoxygenase inhibitor is a 15-lipoxygenase inhibitor; wherein the GPX4 inhibitor is selected from RSL3, ML162, buthionine sulfoximine and a inhibitory nucleic acid molecule.

70-77. (canceled)

78. The method of claim 68, wherein the immunotherapeutic agent is selected from a CTLA4, PDL1 or PD1 inhibitor.

79-81. (canceled)
